(19) United States
(12) Patent Application Publication
(10) Pub. No.: US 2005/0260623 A1
(43) Pub. Date: Nov. 24, 2005

(54) Oct-4 and GJIC expression as markers for adult human stem cells and cancer cell precursors

(75) Inventors: James E. Trosko, Okemos, MI (US); Mei-Hui Tai, East Lansing, MI (US); Chia-Cheng Chang, Okemos, MI (US); L. Karl Olson, East Lansing, MI (US); Burra V. Madhukar, Okemos, MI (US)

Correspondence Address: WILMER CUTLER PICKERING HALE AND DORR LLP, 60 STATE STREET, BOSTON, MA, 02109, US

(73) Assignee: Michigan State University

(21) Appl. No.: 11/069,185

(22) Filed: Feb. 28, 2005

Related U.S. Application Data
(60) Provisional application No. 60/559,747, filed on Apr. 6, 2004.
(60) Provisional application No. 60/548,212, filed on Feb. 27, 2004.

(51) Int. Cl. C12Q 1/68; C12N 5/08

(52) U.S. Cl. 435/6; 435/366; 435/370; 435/369

(57) ABSTRACT

The present invention relates to the identification of adult human stem cells as well as the identification of metastatic cancer cells by detecting the expression of Oct-4, and the lack of GJIC activity. The invention further provides methods of identifying compounds that possess carcinogenic initiator activity, as well as compounds that protect against this earliest stage of cancer.

### CROSS REFERENCE TO RELATED APPLICATIONS

This Application claims the benefit of priority to U.S. Provisional Application No. 60/559,747, filed Apr. 6, 2004, and to U.S. Provisional Application No. 60/548,212, filed Feb. 27, 2004.

This invention was funded in part with a grant from the Michigan Life Sciences Corridor Fund number GR178.

### FIELD OF THE INVENTION

This invention relates to methods and compositions for the detection of expression of certain cell markers for the identification of human adult stem cells and metastatic cells.

### BACKGROUND OF THE INVENTION

Stem cells are a type of reserve cell whose roles is to replace cells that are destroyed during the normal life of the animal, such as blood cells, epithelial cells, and skin cells. Stem cells are thought to be capable of dividing without limit, and can undergo symmetric or asymmetric division to give rise to daughter stem cells, needed for self-renewal and amplification, or daughter progenitor cells, which produce specific differentiated cell lineages (e.g., hematopoietic cell lineages). Given the recent interest in the multiple uses of embryonic and adult stem cells for basic and applied research (e.g., reproductive cloning or regenerative tissue therapy), attempts have been made to characterize markers that would identify these stems cells. While much progress has been made in profiling the expression patterns of hematopoietic stem cells (HSC), neural stem cells (NSC) and embryonic stem cells (ESC) (reviewed in Schepers (2003) Chembiochem 4:716-20), additional markers specific for adult stem cells or specific subpopulations thereof are needed.

Cancer cells can be described as either benign or malignant. Benign cancer cells are generally non-motile. Malignant cancer cells, in contrast, can move from one part of the body to another. This ability to move is called metastasis. Metastatic cancer cells can settle elsewhere in the body and give rise to new tumors. While much progress has been made in identifying markers of metastatic cancer cells, the ability to identify easily and accurately these cells would provide a major advance in the war against cancer.

Accordingly, materials and methods for the identification of adult stem cells as well as the ability to identify malignant cancer cells would be useful in addressing these issues.

### SUMMARY OF THE INVENTION

In one aspect, the invention provides a method of identifying an adult mammalian stem cell in a population of adult mammalian cells by contacting the adult mammalian cell population with an Oct-4 probe; and then identifying cells that express Oct-4. The cells expressing Oct-4 are thereby identified as adult mammalian stem cells. In certain embodiments, the mammalian cells used are human mammalian cells. In further embodiments, the population of adult mammalian cells are from an adult mammalian tissue, which can be, without limitation, breast, liver, pancreas, kidney, mesenchyme or gastric tissue. In still further embodiments, the adult mammalian tissue is from a mammal such as, without limitation, a human, a dog, or a rat.

In certain particularly useful embodiments, the method of identifying adult mammalian stem cells in the population of adult mammalian cells further includes the step of isolating, from the population of mammalian cells, the thus identified adult mammalian stem cell that expresses Oct-4.

In still further embodiments, the method of identifying adult mammalian stem cells in a population of adult mammalian cells further includes the step of detecting GJIC expression or activity in the population of adult mammalian cells. In particular embodiments, the absence of GJIC expression or activity, in an adult mammalian stem cell that expresses Oct-4, further identifies that cell as an adult mammalian stem cell.

In certain useful embodiments, the method of identifying adult mammalian stem cells in the population of adult mammalian cells further includes the step of obtaining a replicate population of the adult mammalian cell population. This allows a replicate sample to be maintained in a viable state while its twin is assayed for Oct-4 and/or GJIC activity. In further embodiments, the replicate population is obtained by isolating individual cells of the adult mammalian cell population, and allowing the isolated individual cells of the adult mammalian cell population to divide at least once to provide two or more replicate cells. This method further includes the step of separating the two or more replicate cells of each of the isolated individual cells so as to provide a replicate population. In particular embodiments, the isolated individual cells of the adult mammalian cell population are allowed to divide at least once in a low calcium cell culture medium. In still further embodiments, the method further includes the step of obtaining an isolated mammalian adult stem cell from a replicate population. In particularly useful embodiments, the isolated adult mammalian stem cell is obtained from the replicate of a cell that express Oct-4. In further embodiments, the method further includes the step of detecting GJIC expression or activity. In this instance, the absence of GJIC expression or activity, in the cell that express Oct-4, further serves to identify the cell that expresses Oct-4, and its replicate, as adult mammalian stem cells.

In further useful embodiments, the mammalian cells from which the adult mammalian stem cells are identified are, without limit, human breast cells, human liver cells, human pancreatic cells, human kidney cells, human mesenchyme cells or human gastric tissue cells.

In another aspect of the invention, it is contemplated that human adult stem cells and metastatic cancer cells can be identified by detecting the presence or absence of two cell markers. It is further contemplated that these markers are Oct-4 and GJIC activity. It is still further contemplated that human adult stem cells and metastatic cancer cells are identified by detecting the presence of Oct-4 and by lack of detectable presence of GJIC activity. And, although the present invention is not limited to any particular theory or mechanism, it is still further contemplated that the presence of Oct-4 and absence of GJIC activity are universal or near universal markers for human adult stem cells and metastatic cancer cells. The use of Oct-4 and GJIC as markers to identify adult stem cells and metastatic cancer cells has not been reported by those knowledgeable in the art.

In another aspect, the invention provides a method of testing for stem cell character or identity by providing a sample of cells; and testing the cells for the presence of Oct-4 gene expression and gap junctional intercellular communication activity. In certain embodiments, the method further includes the step of identifying cells that test positive for Oct-4 gene expression and show an absence of gap junctional intercellular communication activity.

In certain useful embodiments of this method of the invention, the sample of cells is obtained by biopsy. In particular embodiments, sample of cells is derived from a tissue.

In some embodiments of this method of the invention, the cells in the sample of cells are lysed. In particular embodiments, the presence of Oct-4 gene expression is detected by Western blotting, Southern blotting, Northern blotting, immunohisto-chemistry, PCR, RT-PCR, nucleotide sequencing, amino acid sequencing, HPLC, mass spectroscopy, fragment length polymorphism assays, or by enzymatic detection. In other particular embodiments, the gap junctional intercellular communication activity is assessed by the cell-to-cell transfer of Lucifer Yellow dye.

In another aspect, the invention provides a method of testing compounds for their ability to inhibit cell migration, by providing a sample containing cells, at least a portion of which are characterized by the expression of Oct-4 and the absence of gap junctional intercellular communication activity, and exposing the cells to a test compound. By this aspect of the invention, the exposed cells are then tested using an assay for measuring cell migration and compared to control cells not exposed to the test compound. The results of the cell migration assay for the exposed cells is compared to the results of the cell migration assay for the control cells and, if a decrease in migration relative to the control is seen, then the test compound inhibits cell migration.

Many methods for the identification of the presence or absence of Oct-4 and GJIC activity are contemplated by embodiments of the present invention. For example, embodiments of the present invention contemplate that the assay used may be selected for the non-limiting group of western blotting, Southern blotting, northern blotting, immunohistochemistry, PCR, RT-PCR, nucleotide sequencing, amino acid sequencing, high pressure liquid chromatography (HPLC), mass spectroscopy, fragment length polymorphism assays (e.g., RFLP and CFLP assays), DNA chip assays and enzymatic detection (e.g., enzymatic degradation assays) and nucleic acid detection assays such as those described in U.S. Pat. No. 6,673,616, which is incorporated herein by reference.

In another aspect, the invention provides a method of detecting carcinogenic activity in a test compound. This method includes the steps of providing an adult mammalian stem cell expressing Oct-4; contacting the adult mammalian stem cell expressing Oct-4 with the test compound; and allowing the adult mammalian stem cell expressing Oct-4 to grow under conditions that normally promote differentiation and loss of Oct-4 expression. By this method of the invention, the continued expression of Oct-4 under conditions that normally promote differentiation and loss of Oct-4 expression indicates that test compound possesses carcinogenic activity.

In certain embodiments, the method of detecting carcinogenic activity in a test compound further includes the step of detecting GJIC expression or activity in the adult mammalian stem cell. By this embodiment of the method of the invention, the absence of GJIC expression or activity under conditions that normally promote differentiation and loss of Oct-4 expression further indicates that the test compound possesses carcinogenic activity. In some embodiment, the adult mammalian stem cell expressing Oct-4 provided in the method of the invention is a human adult stem cell. In particular embodiments the human adult stem cell provided may be, without limitation, a breast stem cell, a liver stem cell, a pancreatic stem cell, a kidney stem cell, a mesenchyme stem cell or a gastric stem cell.

In yet another aspect, the invention provides a method of screening compounds for anti-cancer activity by providing an adult mammalian stem cell expressing Oct-4 that has been exposed to a carcinogenic agent such that it continues to express Oct-4 activity under differentiating conditions. This carcinogen treated adult mammalian stem cell is further contacted with a test compound, and the expression of Oct-4, under conditions that normally promote differentiation and loss of Oct-4 expression in the adult mammalian stem cell, is monitored. By this method of the invention, the loss of Oct-4 expression in the carcinogen-treated adult mammalian stem cell that has been contacted with the test compound indicates that the test compound possesses anti-cancer activity.

In particular embodiments, the method of screening compounds for anti-cancer activity further includes the step of detecting GJIC expression or activity in the adult mammalian stem cell. By this method of the invention, the presence of GJIC expression or activity in the cells that would otherwise continue to express Oct-4 under conditions that normally promote differentiation and loss of Oct-4 expression further indicates that the test compound possesses anti-carcinogenic activity.

In particular embodiments of this method of screening compounds for anti-cancer activity, the adult mammalian stem cell expressing Oct-4 that are provided are human adult stem cell. In certain embodiments, the human adult stem cell is, without limitation, a breast stem cell, a liver stem cell, a pancreatic stem cell, a kidney stem cell, a mesenchyme stem cell, or a gastric stem cell.

One embodiment of the present invention contemplates drug screens for compounds that, for example, inhibit or reduce the migration of cells (e.g., metastatic cells). In another embodiment of the present invention, it is contemplated that the compounds that inhibit cell migration do so by inhibiting the action of Oct-4 and/or by inducing the production of GJIC activity. The present invention is not limited to any particular mechanism and, therefore, the mechanism by which any identified compounds work is immaterial to practicing the present invention. In yet another embodiment of the present invention, it is contemplated that the screens used for the identification of inhibitors of cell mobility include, but are not limited to, contacting cells suspected of migrating with at least one compound suspected of inhibiting cell migration and then measuring for a reduction in cell migration. The present invention is not limited to any particular amount of reduction. Any amount of reduction in cell migration is contemplated by the present invention. For example, a reduction of at least 25% is contemplated by the present invention. In another embodiment, it is contemplated that cell migration is reduced by at least 50%. In a useful embodiment, cell migration is reduced by at least 90%. It is also contemplated that using the compounds identified by the drug screens of the present invention for the reduction of cell migration are also embodiments of the present invention.

In one embodiment of the present invention, it is contemplated that the drug screens are conducted in vivo and in vitro. For example, in one embodiment, animal models are used to test the migration of, e.g., cells from natural tumors, cells from implanted tumors or implanted migratory cells. One or more compounds suspected of inhibiting cell migration are administered to the animal and cell migration and any inhibition thereof are observed and measured. Cell migration may be measured by, for example, labeling migratory cells with fluorescent tags, radioactive tags or other biochemical agents.

In another embodiment, migratory cells are placed in cell culture conditions known in the art. The present invention is not limited to the type of migratory cell used. In deed, the type of migratory cell used will be determined by the needs of the person(s) conducting the assay. One or more compounds suspected for inhibiting or reducing cell migration are added to the cultures and cell migration is measured. Cell migration may be measured by, for example, labeling migratory cells with fluorescent tags, radioactive tags or other biochemical agents. Additionally, cell migration may be measured by observation. For example, cells could be cultured in a cluster. Migratory cells would be observed to migrate away from the cluster. In conditions where cells were subjected to the agents suspected of inhibiting cell migration, one may see reduced migration as compared to a control culture conditions.

In one embodiment, the present invention contemplates that Oct-4 and connexin peptides and nucleic acids may be used for therapeutic uses in, for example, the control of cell migration. In a particular embodiment, the present invention contemplates that antisense technology is used. In another embodiment, it is contemplated that viral vectors are used to introduce the therapeutic peptides and nucleic acids that are delivered to cells in the patient. In yet another embodiment, it is contemplated that the viral vectors are directed to target cells via interactions with cell specific receptors. In a useful embodiment, the viral vectors used are replication defective.

The invention also relates to methods to identify other binding partners of the Oct-4 and connexin genes and gene products. These binding partners could be used, for example, for therapeutic or research purposes. The present invention is not limited to the methods employed to identify Oct-4 and connexin genes and gene product binding partners. In one embodiment, antibodies generated to translation products of the invention may be used in immunoprecipitation experiments to isolate novel Oct-4 and connexin genes and gene product binding partners or natural mutations thereof. In another embodiment, the invention may be used to generate fusion proteins (e.g. Oct-4 and connexin fusion proteins) that could also be used to isolate novel Oct-4 and connexin genes and gene product binding partners or natural mutations thereof. In yet another embodiment, screens may be conducted using the yeast two-hybrid system using Oct-4 or connexin as the bait. In yet another embodiment, screens may be conducted using affinity chromatography using Oct-4 or connexin as the ligand.

The invention also relates to the production of derivatives of the Oct-4 and connexin genes such as, but not limited to, mutated gene sequences (and portions thereof), transcription products (and portions thereof), expression constructs, transfected cells and transgenic animals generated from the nucleotide sequences (and portions thereof). The present invention also contemplates antibodies (both polyclonal and monoclonal) to the gene product or nucleic acid aptamers (i.e., an oligonucleotide capable of binding with a target molecule such as an antibody), including the product of mutated genes.

In one embodiment, the present invention contemplates a testing method, comprising: a) providing a sample comprising cells; b) testing said cells for the presence of Oct-4 gene expression and gap junctional intercellular communication function. In another embodiment, the present invention contemplates that method further comprises) identifying cells which test positive for Oct-4 gene expression and show an absence of gap junctional intercellular communication (GJIC) activity. In yet another embodiment, the present invention contemplates that the amount of GJIC activity is no more that two times above background. In still yet another embodiment, the present invention contemplates that the amount of GJIC activity is no more than 50% of the positive control. In still yet another embodiment, the present invention contemplates that the amount of GJIC activity is no more than 25% of the positive control. In still yet another embodiment, the present invention contemplates that the amount of GJIC activity is no more than 10% of the positive control. In still yet another embodiment, the present invention contemplates that the sample is obtained by biopsy. In still yet another embodiment, the present invention contemplates that the sample comprises tissue. In still yet another embodiment, the present invention contemplates that prior to step b) said cells are lysed. In still yet another embodiment, the present invention contemplates that the testing for the presence of Oct-4 gene expression comprises methodology selected from the group consisting of Western blotting, Southern blotting, Northern blotting, immunohistochemistry, PCR, RT-PCR, nucleotide sequencing, amino acid sequencing, HPLC, mass spectroscopy, fragment length polymorphism assays, and enzymatic detection. In still yet another embodiment, the present invention contemplates that the testing for gap junctional intercellular communication function comprises testing for the transfer of Lucifer Yellow dye from cell-to-cell.

In one embodiment, the present invention contemplates that a method of testing compounds for inhibition of cell migration, comprising: a) providing: i) a sample comprising cells, at least a portion of said cells characterized by the expression of Oct-4 and the absence of gap junctional intercellular communication activity, ii) a test compound, and iii) an assay for measuring cell migration; b) exposing said cells to said test compound to create treated cells; and c) assaying said treated cells with said migration assay. In another embodiment, the present invention contemplates that the assaying of step c) comprises comparing the migration of said treated cells in said migration assay with one or more controls to detect inhibition of cell migration. In another embodiment, the present invention contemplates that the amount of GJIC activity is no more that two times above background. In yet another embodiment, the present invention contemplates that the amount of GJIC activity is no more than 50% of the positive control. In still yet another embodiment, the present invention contemplates that the amount of GJIC activity is no more than 25% of the positive control. In still yet another embodiment, the present invention contemplates that the amount of GJIC activity is no more than 10% of the positive control.

### BRIEF DESCRIPTION OF THE FIGURES

FIG. 1(A) is a photographic representation of a fluorescent-microscopic image showing Oct-4 protein expression in human breast stem cells. Inset of A shows the punctate Oct-4 staining located in the nucleus as double labeled with 4′,6′-diamidino-2-phenylindole (DAPI).

FIG. 1(A1) is a photographic representation of a phase-contrast image showing the morphology of the cells in FIG. 1(A).

FIG. 1(B) is a photographic representation of a fluorescent-microscopic image showing Oct-4 protein expression in human breast stem cells. Inset of B shows that Oct-4 staining is weak or missing in Type II human breast differentiated cells.

FIG. 1(B1) is a photographic representation of a phase-contrast image showing the morphology of the cells in FIG. 1(B).

FIG. 1(C) is a photographic representation of a fluorescent-microscopic image showing Oct-4 protein expression in differentiating human breast stem cells. Inset of C shows that Oct-4 staining of one colony of human breast stem cells in the transition of differentiation into differentiated cells.

FIG. 1(C1) is a photographic representation of a phase-contrast image showing the morphology of the cells in FIG. 1(C).

FIG. 2(A) is a photographic representation of a fluorescent-microscopic image showing Oct-4 protein expression in a human breast immortal, tumorigenic cell line. Inset of A shows a higher magnification image of double labeled of Oct-4 and DAPI double labeled cells. The punctate Oct-4 staining located in the nucleus as double labeled with DAPI (blue).

FIG. 2(A1) is a photographic representation of a phase-contrast image showing the morphology of the cells in FIG. 2(A).

FIG. 2(B) is a photographic representation of a fluorescent-microscopic image showing Oct-4 protein expression in a human breast immortal, tumorigenic cell line. Inset of B shows a higher magnification image of double labeled of Oct-4 and DAPI double labeled cells. The punctate Oct-4 staining located in the nucleus as double labeled with DAPI (blue).

FIG. 2(B1) is a photographic representation of a phase-contrast image showing the morphology of the cells in FIG. 2(B).

FIG. 2(C) is a photographic representation of a fluorescent-microscopic image showing Oct-4 protein expression in a human breast immortal, tumorigenic cell line. Inset of C shows a higher magnification image of double labeled of Oct-4 and DAPI double labeled cells. The punctate Oct-4 staining located in the nucleus as double labeled with DAPI (blue).

FIG. 2(C1) is a photographic representation of a phase-contrast image showing the morphology of the cells in FIG. 2(C).

FIG. 2(D) is a photographic representation of a reverse transcription-PCR assay of total RNA extracted from cell cultures, of human breast immortal, weakly tumorigenic and highly tumorigenic cell lines, using primer sets designed to amplify fragments of Oct-4 (top) and GAPDH (positive control, bottom). Lane 1-2: human breast stem cells showing positive Oct-4 expression. Lane 3: human breast stem cells in transitional into differentiated cells showing decreased but persist Oct-4 expression. Lane 4-6: human breast immortal, weakly tumorigenic and highly tumorigenic cell lines showing very strong Oct-4 expression. Lane 7-8: negative control (no template).

FIG. 3(A) is a photographic representation of a low magnification fluorescent-microscopic image showing Oct-4 protein expression in human pancreatic stem cells cultured in proliferation medium (KNC medium which is low calcium medium plus anti-oxidant reagents, N-acetyl-L-cysteine and ascorbic acid). Cells were fixed and double-labeled with DAPI for DNA and Oct-4.

FIG. 3(A1) is a photographic representation of a phase-contrast image showing the morphology of the cells in FIG. 3(A).

FIG. 3(B) is a photographic representation of a higher magnification fluorescent-microscopic image showing Oct-4 protein expression in human pancreatic stem cells cultured in proliferation medium (KNC medium which is low calcium medium plus anti-oxidant reagents, N-acetyl-L-cysteine and ascorbic acid). Cells were fixed and double-labeled with DAPI for DNA and Oct-4. The punctate red staining indicates Oct-4 protein expression in the pancreatic stem cells. The inset shows the merged image of FIG. 3(B) and FIG. 3(B1).

FIG. 3(B1) is a photographic representation of a higher magnification fluorescent-microscopic image of DAPI staining in showing the nuclear location of the cells in FIG. 3(B).

FIG. 3(C) is a photographic representation of a higher magnification fluorescent-microscopic image showing Oct-4 protein expression in human pancreatic differentiated daughter cells. Cells were fixed and double-labeled with DAPI for DNA and Oct-4. The image show that Oct-4 expression is decreased in human pancreatic cells cultured in the differentiation medium (neuronal medium plus N2 supplement, LY294002 and nicotinamide).

FIG. 3(C1) is a photographic representation of a higher magnification phase contrast image of the cells shown in in FIG. 3(C).

FIG. 3(D) is a photographic representation of a reverse transcription-PCR assay of total RNA extracted from cell cultures, subject to different medium conditions, using primer sets designed to amplify fragments of Insulin mRNA (top) and GAPDH mRNA (positive control, bottom). Lane 1-2: human pancreatic stem cells cultured in the proliferation medium (KNC) showing no insulin message. Lane 3-4: human pancreatic stem cells were cultured in the differentiation medium showing insulin message. Lane 5: positive control of insulin expression of human islet RNA. Lane 6: negative control (no template). Oct-4 expression level in cells cultured in the differentiation medium also decreased compared to the cells cultured in the proliferation medium (data not shown).

FIG. 4(A) is a photographic representation of a fluorescent-microscopic image showing Oct-4 protein expression in the human pancreatic cancer cell line Capan-2.

FIG. 4(A1) is a photographic representation of a phase-contrast image showing the morphology of the cells in FIG. 4(A).

FIG. 4(B) is a photographic representation of a fluorescent-microscopic image showing Oct-4 protein expression in the human pancreatic cancer cell line Pan-1.

FIG. 4(B1) is a photographic representation of a phase-contrast image showing the morphology of the cells in FIG. 4(B).

FIG. 5(A) is a photographic representation of a fluorescent-microscopic image showing nuclear punctate staining of Oct-4 in human liver stem cells.

FIG. 5(A1) is a photographic representation of a fluorescent-microscopic image showing DAPI nuclear staining of the cells in FIG. 5(A).

FIG. 5(B) is a photographic representation of a fluorescent-microscopic image showing that Oct-4 is not visible in differentiated liver cells.

FIG. 5(B1) is a photographic representation of a phase-contrast image showing the morphology of the cells in FIG. 5(B).

FIG. 5(C) is a photographic representation of a fluorescent-microscopic image showing the expression of Oct-4 protein in SV40-immoralized liver cells.

FIG. 5(C1) is a photographic representation of a phase-contrast image showing the morphology of the cells in FIG. 5(C).

FIG. 5(D) is a photographic representation of a fluorescent-microscopic image showing nuclear punctate staining of Oct-4 in a human liver tumor cell line.

FIG. 5(D1) is a photographic representation of a fluorescent-microscopic image showing DAPI nuclear staining of the cells in FIG. 5(D).

FIG. 6(A) is a photographic representation of a low-magnification fluorescent-microscopic image showing Oct-4 protein expression in human mesenchymal stem cells.

FIG. 6(A1) is a photographic representation of a phase-contrast image showing the morphology of the cells in FIG. 6(A).

FIG. 6(B) is a photographic representation of a high-magnification fluorescent-microscopic image showing nuclear punctate staining of Oct-4 in human mesenchymal stem cells.

FIG. 6(B1) is a photographic representation of a high-magnification fluorescent-microscopic image showing DAPI nuclear staining of the cells in FIG. 6(B).

FIG. 7(A) is a photographic representation of a low-magnification fluorescent-microscopic image showing Oct-4 protein expression in human kidney stem cells.

FIG. 7(A1) is a photographic representation of a low-magnification phase-contrast image showing the morphology of the cells in FIG. 7(A).

FIG. 7(B) is a photographic representation of a high-magnification fluorescent-microscopic image showing nuclear punctate staining of Oct-4 in human kidney stem cells.

FIG. 7(B1) is a photographic representation of a high-magnification fluorescent-microscopic image showing DAPI nuclear staining of the cells in FIG. 6(B).

FIG. 7(C) is a photographic representation of a low-magnification fluorescent-microscopic image showing Oct-4 protein expression in human gastric stem cells.

FIG. 7(C1) is a photographic representation of a low-magnification phase-contrast image showing the morphology of the cells in FIG. 7(C).

FIG. 8(A) is a photographic representation of a fluorescent-microscopic image showing Oct-4 protein expression in HeLa cells cultured in the differentiation D medium. The arrow shows a colony in the center showing very intense Oct-4 expression in the nucleus of the cells compared to no expression of Oct-4 of the cells on the side of the image.

FIG. 8(A1) is a photographic representation of a phase-contrast image showing the morphology of the cells in FIG. 8(A).

FIG. 8(B) is a photographic representation of a fluorescent-microscopic image showing sparse MCF7 cells exhibiting Oct-4 protein expression when cultured in the differentiation D medium.

FIG. 8(B1) is a photographic representation of a phase-contrast image showing the morphology of the cells in FIG. 8(B).

FIG. 8(C) is a photographic representation of a reverse transcription-PCR assay of total RNA extracted from different stem cells, immortalized and cancer cell lines using primers designed to amplify fragments of Oct-4 (top) and GAPDH (positive control, bottom). Lane 1: human pancreatic stem cells. Lane 2: immortalized pancreatic cell line. Lane 3 and 4: human pancreatic cancer cell lines, Capan-2 and Pan-1 cell lines. Lane 5: human liver stem cells. Lane 6: human liver cancer cell line. Lane 7: human mesenchymal stem cells. Lane 8: human gastric stem cell. Lane 9: immortalized human gastric cell lines. Lane 10: human kidney stem cells. Lane 11-12: HeLa and MCF7 cell lines. Lane 13: negative control (no template).

FIG. 9(A) is a photographic representation of a microscopic image showing Oct-4 expression in the basal layer of a normal human tissue thin section. The normal human skin thin section was de-paraffinized and stained with Oct-4 primary antibody and avidin-HRP, and then visualized with DAB.

FIG. 9(A1) is a photographic representation of a high-magnification microscopic image of the stained section shown in FIG. 9(A). The arrow indicates the nuclear localization of Oct-4 expression in an Oct-4 expressing cell.

FIG. 9(B) is a photographic representation of a microscopic image showing Oct-4 expression in the basal layer of a dog tissue thin section. The dog skin thin section was de-paraffinized and stained with Oct-4 primary antibody and avidin-HRP, and then visualized with DAB.

FIG. 9(B1) is a photographic representation of a high-magnification microscopic image of the stained section shown in FIG. 9(B). The arrow indicates the nuclear localization of Oct-4 expression in an Oct-4 expressing cell.

FIG. 10(A) is a schematic representation of the polypeptide sequence of a human Oct-4 corresponding to GenBank Accession No. Q01860 (SEQ ID NO: 7).

FIG. 10(B) is a schematic representation of the nucleotide sequence of a human Oct-4-encoding nucleic acid sequence corresponding to GenBank Accession No. Z11898 (SEQ ID NO. 5). The initiation and termination codons of the Oct-4 protein open reading frame are underlined.

FIG. 11(A) is a schematic representation of the polypeptide sequence of a human connexin 43 corresponding to GenBank Accession No. NP_{—}000156 (SEQ ID NO: 8).

FIG. 11(B) is a schematic representation of the nucleotide sequence of a human connexin 43-encoding nucleic acid sequence corresponding to GenBank Accession No. NM_{—}000165 (SEQ ID NO. 6). The initiation and termination codons of the connexin 43 protein open reading frame are underlined.

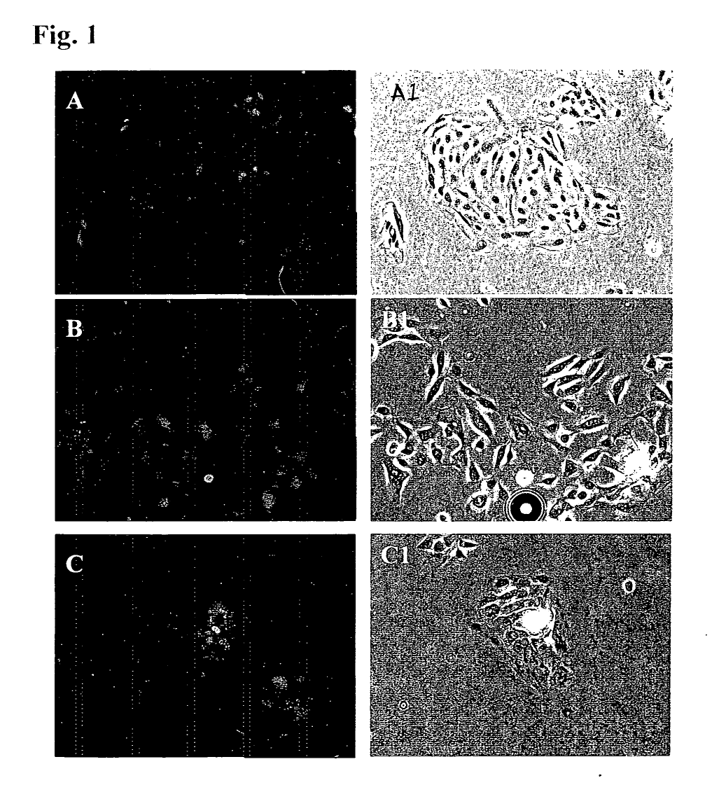

### DETAILED DESCRIPTION OF THE INVENTION

The patent and scientific literature referred to herein establishes knowledge that is available to those of skill in the art. The issued U.S. patents, allowed applications, published foreign applications, and references, including GenBank database sequences, which are cited herein are hereby incorporated by reference to the same extent as if each was specifically and individually indicated to be incorporated by reference. This Application incorporates by reference in their entireties the teachings of U.S. Provisional Application No. 60/577,815, filed Jun. 8, 2004, entitled “Adult Stem Cells and Uses Thereof”, and U.S. Provisional Application No. 60/548,340, filed Feb. 27, 2004, entitled “Clonal Adult Liver Stem Cells”.

4.1. General

The invention is based, in part, on the finding that Oct-4, and certain other markers, are particular hallmarks of stem cells present in normal adult mammalian tissues. While the expression and regulation of Oct-4 in the early mammalian embryo has been investigated (see, e.g., Pesce and Scholer (2001) Stem Cells 19: 271-8), these reports have widely held that Oct-4 gene expression outside of the germ cell lineage is limited to the blastocyst and gastrulating early embryo and that Oct-4 is not expressed in adult human tissue (Mitsui et al. (2003) Cell 113: 631-42). The invention thereby provides a unique marker for identifying adult human stem cells for use in innumerable regenerative applications.

Furthermore, the invention provides an additional practical insight into the etiology of human cancers, and thereby supports multiple novel methods for the screening of, e.g., cancer preventives and early cancer diagnostics. In particular, the prevailing paradigm in the cancer field is that a normal cell must be a “mortal” cell that must first be “immortalized,” and then subsequently must be neoplatically transformed (see Trosko, J. E., et al., “Oncogenes, tumor suppressor genes and intercellular communication in the ‘Oncogeny as partially blocked ontogeny’ hypothesis”. In: New Frontiers in Cancer Causation, Ivrersen, O. H., ed., Taylor and Francis Publisher, Washington, D.C., pp. 181-187, 1993). This is called the “de-differentiation” theory of carcinogenesis. An alternate theory of carcinogenesis is called the “stem cell” theory. This theory proposes that adult stem cells, which, by their nature, are immortal, lose growth regulation and, thus, become carcinogenic. While the present invention is not limited to any particular mechanism, the embodiments of the present invention lend considerable support to this theory. In this regard, some embodiments of the present invention provide materials and methods for the identification of adult stem cells and metastatic cancer cells: namely, the identification of such cells by the expression of Oct-4 and the lack of GJIC activity.

Oct-4

The present invention is not limited to any particular theory or mechanism. In fact, an understanding of the theory or mechanism is not needed to practice the present invention.

The transcription factor variously known as Oct-3, Oct-4 and Oct-3/4 was discovered in the early nineties almost simultaneously by (in order of appearance) Okamoto, et al., (1990) Cell, 461-472, Schöler, et al., (1990) and Rosner, et al., (1990) Nature, 345: 686-692. The gene coding for this transcription factor is now known as Pou5f1. Regardless of its name (for this application it will be called Oct-4), it plays an important role in development.

Gap Junctions

In contrast to tight and adherens junctions, gap junctions do not seal membranes together, nor do they restrict the passage of material between membranes. Rather, gap junctions are composed of arrays of small channels that permit small molecules to shuttle from one cell to another and thus directly link the interior of adjacent cells. Importantly, gap junctions allow electrical and metabolic coupling among cells because signals initiated in one cell can readily propagate to neighboring cells. In general, the upper limit for passage through gap junctions is roughly 1000 daltons (Da). Aside from ions, important examples of molecules that readily pass include cyclic AMP (329 Da), glucose-6-phosphate (259 Da) and nucleotides (250-300 Da).

Gap junctions are seen with an electron microscope as patches of varying size where the plasma membranes of neighboring cells are separated by a beautifully uniform gap, roughly 2-3 nm in width. The gap reflects areas of the two membranes that are connected by hexagonal tubes called connexons which form aqueous pores roughly 2 nm in diameter between the two cells. The major protein in purified preparations of gap junctions is connexin (e.g., connexin 43, which, when expressed in cells that normally do not have gap junctions, allows them to form. Different species of connexin are seen in different organisms and among different tissues within an organism. However, all connexins share a common structure of having four membrane-spanning domains. A connexon is formed from six connexin molecules that extend a uniform distance outside the cells. Alignment of connexons from each cell across the gap results in the formation of the pores that functionally define the gap junction.

Gap junctions are dynamic structures because connexons are able to open and close. Elevated intracellular calcium and low intracellular pH are established stimuli for rapid closing of connexons. This may be of importance when one cell within a group becomes damaged; the idea is that closing the gap junctions in the damaged cell would effectively isolate that cell and prevent spreading of the injury. Gap junctions are seen in virtually all cells that contact other cells in tissues that, of course, includes pretty much most cells in the body.

Stem Cells and Cancer Cells

Stem cells exist in most, if not all, adult organs. They are defined as cells that undergo symmetric and asymmetric division to give rise to daughter cells needed for self-renewal and amplification or for giving rise to a daughter cell that acts as a progenitor or transit cell for the purpose of producing specific differentiated lineages, respectively. Given the recent interest in the multiple uses of embryonic and adult stem cells for basic and applied research (i.e., reproductive cloning or regenerative tissue therapy), attempts have been made to characterize markers that would identify these stems cells.

Oct3/4 or Oct4 (now referred to as Pou5f1), a transcription factor, was discovered in 1990 (Okamoto, et al., (1990) Cell, 461-472; Scholer, et al., (1990) Nature 344: 435-439; and Rosner, et al., (1990) Nature, 345: 686-692). This factor was found in ovulated oocytes, mouse pre-implantation embryos, ectoderm of the gastrula (but not in other germ layers), primordial germ cells, as well as in embryonic stem cells but not in their differentiated daughters (Solter, 2002). More recently, OCT4/Pou5f1 has been shown in cells isolated from human amniotic fluid (Prusa, et al., (2003) Hum. Reprod., 18:1489-1493), as well as a useful marker for pluripotency after activation of the embryonic genome (Mitalipov, et al., 2003). Subsequent studies seemed to suggest that Oct4 or Pou5f1 might be a specific expressed gene marker for totipotency or a gene required for imposing totipotency (Pesce, et al., (1998) Bioessays, 20:722-732; Pesce and Scholer, (2001) Stem Cells, 19:271-278.

On the other hand, the gene has been shown to be expressed in human tumor cells that have been tested but not to be expressed in normal somatic tissues (Monk, et al., (2001) Oncogene, 20:8085-8091). Among some “hallmarks” of cancer cells (Hanahan, et al., (2000) Cell, 100:57-70) is the ability to have indefinite proliferative potential. In addition, cancer cells do not have functional homologous or heterologous gap junctional intercellular communication (GJIC) (Yamasaki, H., et al., Cancer Detect. Prev. 23:273-279 (1999), due either to the non-expression of connexins (e.g., HeLa and MCF-7 cells) or to the non-function of expressed connexins (tumor cells expressing ras-; src-; or neu-oncogenes) (Trosko, et al., (1998) Front. Biosci. 3:208-236). Gap junctions have been associated with both normal development (Lo, C. and M. Delmar, Cell. Commun. Adhes. 10:169-172 (2003), growth control, differentiation, wound repair, synchronization of metabolic secretion and electrotonic function in tissues (Evans, et al., Mol. Membr. Biol., 19:121-136; TenBroek, et al. (2001), J. Cell Biol., 155:1307-1318. Interestingly, several isolated presumptive adult human stem cells have been characterized as being deficient in the expression of connexins and gap junctional intercellular communication (Human kidney epithelial stem cells: Chang, C. C., et al., Cancer Res. 47:1734-1645, 1987; Human breast epithelial stem cells: Kao, C. Y., et al., Carcinogenesis 16:531-538, 1995; Human pancreas: Tai, et al., (2003), Pancreas, 26:e18-e26; Human keratinocyte: Matic, et al., (2002), J. Invest. Dermatol., 118:110-116; Human corneal epithelium cells; Matic, et al., (1997), Differentiation, 61:251-260.

4.2 Definitions

“Oct-4,” “Oct-3,” “Oct-3/4” and “pou5f1” are considered the same in the literature (Solter, D, “Cloning v. Clowning” Genes & Dev. 16:1163-1166, 2002) and will be considered to the same in the present application. Oct-4 is a gene that encodes a transcription factor that is believed to play a role during development (see Okamoto, et al. (1990) Cell 60: 461-72; Rosner, et al. (1990) Nature 344: 435-9; Schöler, et al. (1990) Nature 345: 686-92).

“Connexin(s)” (e.g., connexin 43, connexin 45, connexin 32, connexin 26 and connexin 37) are proteins associated with “Gap Junction Intercellular Communication” (GJIC) activity. “Gap junctions” are protein complexes that function in the adherence, communication, growth and growth inhibition of cells. For more information on gap junctions, see, The General Description of the Invention: Gap Junctions, infra. In the present application the terms “connexin(s)” shall refer to all connexins incorporated into gap junctions.

The term “connexon” refers to the functional unit of gap junctions. It's an assembly of six membrane spanning proteins (connexins) having a water filled gap in the center. Two connexons in juxtaposed membranes link to form a continuous pore through both membranes (ie., a gap junction).

The present invention contemplates that “testing” (or assaying) of, for example, Oct-4 expression and GJIC activity may be sequential, parallel, simultaneous or substantially simultaneous. Embodiments of the present invention are not limited to the timing of the testing. Such tests may be conducted simultaneously, substantially simultaneously, sequentially or in parallel. Simultaneous, substantially simultaneous, sequential and parallel tests may take place in the same reaction vessel or in different reaction vessels. Substantially simultaneous testing refers to testing that overlaps with other testing. That is, for example, one test can start before or after another test or end before or after another test. If the first test has completed before the second test commences or at about the same time the second test commences then the testing is sequential. Sequential testing is not limited to any particular time frame. For example, the tests may be run immediately one after the other or there may be a delay between tests. The length of the delay is immaterial to embodiments of the present invention so long as the results of the tests are not affected materially. The length of the delay may also be influenced by other considerations such as storage conditions of the sample. For example, refrigeration or freezing of the sample would increase the allowable time of the delay substantially. In one embodiment, it is contemplated that the sequential tests are performed within one year of each other. In a more useful embodiment, the tests are performed with in one month of each other. In a more useful embodiment, the tests are performed with in one week of each other. In a most useful embodiment, the tests are performed with in 48 hours of each other.

The phrase “absence of gap junction intracellular communication activity” (and similar phrases) shall not limit the embodiments of the present invention to the total absence or total detectable absence of GJIC activity. Rather, “absence of gap junction intracellular activity” refers to, for example, reduced GJIC activity as compared to control samples and it can refer to a percentage of GJIC activity as compared to control samples (e.g., less than 20% GJIC activity of the positive control sample). In yet another embodiment, the present invention contemplates that the amount of GJIC activity is no more that two times above background. In still yet another embodiment, the present invention contemplates that the amount of GJIC activity is no more than 50% of the positive control. In still yet another embodiment, the present invention contemplates that the amount of GJIC activity is no more than 25% of the positive control. In still yet another embodiment, the present invention contemplates that the amount of GJIC activity is no more than 10% of the positive control. Additionally, one method of detection of GJIC activity may detect minimal amounts of activity while another method of detection of GJIC activity may not detect the same minimal amounts of GJIC activity. As such, embodiments of the present invention are not limited to any particular method of detecting GJIC activity. In fact, many exemplary methods are listed below in the Detailed Description of Useful Embodiments section.

The terms “protein,” “peptide” and “polypeptide” refer to compounds comprising amino acids joined via peptide bonds and are used interchangeably. A “protein,” “peptide” or “polypeptide” encoded by a gene is not limited to the amino acid sequence encoded by the gene, but includes post-translational modifications of the protein.

The term “metastatic” shall refer to the spread of a disease from the organ or tissue of origin to another part of the body. In regards to cancer, the cells that spread the disease are called “metastatic cells.”

The term “cell migration” shall refer to the ability of cells to move from one place to another in, for example, a body or in a culture condition. The term “assay for measuring cell migration” (and similar phrases) shall refer to biological or biochemical methods for detecting the movement of a cell of group of cells from one location to one or more new locations. For example, the assay may biochemically measure the movement of tagged cells through the body or visually measure the movement of cells in a culture condition (e.g., a petri dish).

The term “patient” shall refer to an individual (e.g., a person or other mammal) who is receiving medical treatment (accepted by the standards of care by those in the profession or experimental). The term patient shall also refer to research animals. For the purposes of this application, the term “subject” shall be synonymous with the term patient.

Where the term “amino acid sequence” is recited herein to refer to an amino acid sequence of a protein molecule, “amino acid sequence” and like terms, such as “polypeptide,” “peptide” or “protein”, are not meant to limit the amino acid sequence to the complete, native amino acid sequence associated with the recited protein molecule. Furthermore, an “amino acid sequence” can be deduced from the nucleic acid sequence encoding the protein. Detecting amino acids sequences encoded by the Oct-4 (for example, GenBank Accession Number Q01860; SEQ ID NO: 7) or Connexin 43 (for example, GenBank Accession Number NP_{—}000156; SEQ ID NO: 8) genes or portions thereof is contemplated by one embodiment of the present invention. (In other embodiments, the nucleic acid sequences encoding Oct-4 and connexin 43 are detected—SEQ IN NO: 5 (GenBank Accession No. Z11898) and SEQ ID NO: 6 (GenBank Accession No. NM_{—}000165) correspond to the Oct-4 and connexin 43 genes respectively).

The term “portion” when used in reference to a protein (as in “a portion of a given protein”) refers to fragments of that protein. The fragments may range in size from four amino acid residues to the entire amino sequence minus one amino acid. The term “portion” when used in reference to a nucleic acid (as in “a portion of a given nucleic acid”) refers to fragments of that nucleic acid. The fragments may range in size from ten bases to the entire nucleic acid sequence minus one base.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms.

In one embodiment of the present invention it is contemplated that exogenous genes will be used to control the endogenous and exogenous expression of Oct-4 and connexins. The expressed exogenous proteins may be part of a fusion protein. The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The term “homolog” or “homologous” when used in reference to a polypeptide refers to a high degree of sequence identity between two polypeptides, or to a high degree of similarity between the three-dimensional structures or to a high degree of similarity between the active site and the mechanism of action. In a useful embodiment, a homolog has a greater than 60% sequence identity, and more usefully greater than 75% sequence identity, and still more usefully greater than 90% sequence identity, with a reference sequence.

As applied to polypeptides, the term “substantial identity” means that two peptide sequences, when optimally aligned, such as by the programs GAP or BESTFIT using default gap weights, share at least 80% sequence identity, usefully at least 90% sequence identity, more usefully at least 95% sequence identity or more (e.g., 99% sequence identity). Usefully, residue positions that are not identical differ by conservative amino acid substitutions.

In one embodiment of the present invention it is contemplated that variants of the Oct-4 peptide or variants of various gap junction proteins (e.g., connexins and more specifically, for example, connexin 43) may be used for, e.g., transfections. The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Useful conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Useful variants have less than 10%, usefully less than 5% and still more usefully less than 2% changes (whether substitutions, deletions, and so on).

The term “domain” when used in reference to a polypeptide refers to a subsection of the polypeptide that possesses a unique structural and/or functional characteristic; typically, this characteristic is similar across diverse polypeptides. The subsection typically comprises contiguous amino acids, although it may also comprise amino acids that act in concert or which are in close proximity due to folding or other configurations.

In one embodiment of the present invention contemplates several genes (e.g., Oct-4 (SEQ ID NO: 5) and Connexin 43 (SEQ ID NO: 6). The term “gene” refers to a nucleic acid (e.g., DNA or RNA) sequence that comprises coding sequences necessary for the production of a RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

In one embodiment of the present invention it is contemplated that the genes of the present invention comprise introns and exons. A genomic form or clone of a gene contains the coding region interrupted with non-coding sequences termed “introns” or “intervening regions” or “intervening sequences.” Introns are segments of a gene that are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

In one embodiment of the present invention, it is contemplated that the nucleic acids encoding the Oct-4 and connexin 43 or another connexin peptides may be expressed in organisms or cells wherein they are not normally expressed. The term “heterologous,” when used in reference to a gene, refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise, e.g., plant or animal gene sequences that comprise cDNA forms of a plant or animal gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed). In the present invention, it is contemplated that the nucleotide sequence that encodes Oct-4 and portions thereof may comprise a heterologous gene. For example, the Oct-4 sequence may be joined to promoter specific for various tissues. The present invention is not limited to any particular tissues.

In one embodiment of the present invention, it is contemplated that a portion of the Oct-4 or connexin 43 nucleic acid sequence (i.e., a “nucleic acid sequence of interest”) may be used. The nucleic acid sequences encoding Oct-4 and connexin 43 include, for example, SEQ IN NO: 5 (GenBank Accession No. Z11898) and SEQ ID NO: 6 (GenBank Accession No. NM_{—}000165) respectively.

The term “nucleotide sequence of interest” or “nucleic acid sequence of interest” refers to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “structural” when used in reference to a gene or to a nucleotide or nucleic acid sequence refers to a gene or a nucleotide or nucleic acid sequence whose ultimate expression product is a protein (such as an enzyme or a structural protein), an rRNA, an sRNA, a tRNA, etc.

The terms “oligonucleotide” or “polynucleotide” or “nucleotide” or “nucleic acid” refer to a molecule comprised of two or more deoxyribonucleotides or ribonucleotides, usefully more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The terms “an oligonucleotide having a nucleotide sequence encoding a gene” or “a nucleic acid sequence encoding” a specified polypeptide refer to a nucleic acid sequence comprising the coding region of a gene or in other words the nucleic acid sequence which encodes a gene product. The coding region may be present in either a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

In one embodiment of the present invention, it is contemplated that recombinant techniques are used with the nucleic acid sequences of the present invention. The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence,” “sequence identity,” “percentage of sequence identity” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20% or less as compared to the reference sequence (which does not comprise additions or deletions) for optimal alignment of the two sequences. Optimal alignment of sequences for aligning a comparison window may be conducted by the local homology algorithm of Smith and Waterman (Adv. Appl. Math. 2: 482 (1981) by the homology alignment algorithm of Needleman and Wunsch (J. Mol. Biol. 48:443 (1970), by the search for similarity method of Pearson and Lipman (Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, usefully at least 90 to 95% sequence identity, more usually at least 99 percent sequence identity as compared to a reference sequence over a comparison window of at least 20 nucleotide positions, or over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985). Other references include more sophisticated computations that take structural as well as sequence characteristics into account for the calculation of Tₘ.

In one embodiment of the present invention, it is contemplated that assays will be used for detecting nucleic acids with, for example, labeled probes. In this regard, complementary sequences will hybridize to each other. Hybridization may occur at different stringencies. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄(H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄(H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄(H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

It is well known that numerous equivalent conditions may be employed to comprise low stringency conditions: factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is often the gene that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identifiable since they have altered characteristics when compared to the wild-type gene or gene product.

Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The term “polymorphic locus” refers to a genetic locus present in a population that shows variation between members of the population (i.e., the most common allele has a frequency of less than 0.95). Thus, “polymorphism” refers to the existence of a character in two or more variant forms in a population. A “single nucleotide polymorphism” (or SNP) refers a genetic locus of a single base that may be occupied by one of at least two different nucleotides. In contrast, a “monomorphic locus” refers to a genetic locus at which little or no variations are seen between members of the population (generally taken to be a locus at which the most common allele exceeds a frequency of 0.95 in the gene pool of the population).

A “frameshift mutation” refers to a mutation in a nucleotide sequence, usually resulting from insertion or deletion of a single nucleotide (or two or four nucleotides) that results in a change in the correct reading frame of a structural DNA sequence encoding a protein. The altered reading frame usually results in the translated amino-acid sequence being changed or truncated. The AS-193 variant of the present invention is believed to have a frameshift mutation that produces a premature stop codon after amino acid 416.

A “splice mutation” refers to any mutation that affects gene expression by affecting correct RNA splicing. Splicing mutation may be due to mutations at intron-exon boundaries that alter splice sites.

The term “detection assay” refers to an assay for detecting the presence or absence of a sequence or a variant nucleic acid sequence (e.g., mutation or polymorphism in a given allele of a particular gene, as e.g., Oct-4 gene), or for detecting the presence or absence of a particular protein (e.g., Oct-4) or the structure or activity or effect of a particular protein (e.g., a binding assay or activity assay) or for detecting the presence or absence of a variant of a particular protein.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

In one embodiment of the present invention, it is contemplated that the nucleotide sequences of the present invention may be “amplified”. “Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. Examples of amplification include, but are not limited to, PCR and the INVADER® assay (Third Wave Technologies, Madison Wis.).

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian, et al., Proc. Natl. Acad. Sci. USA, 69:3038 (1972). Other nucleic acids will not be replicated by this amplification enzyme. Similarly, in the case of T7 RNA polymerase, this amplification enzyme has a stringent specificity for its own promoters (Chamberlain, et al., Nature, 228:227 (1970). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 (1989). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

The term “amplifiable nucleic acid” refers to nucleic acids that may be amplified by any amplification method. It is contemplated that “amplifiable nucleic acid” will usually comprise “sample template.” Examples of amplification include, but are not limited to, PCR and the INVADER® assay (Third Wave Technologies, Madison Wis.).

Allele specific nucleic acid sequences may also be identified by hybridization with crosslinkable oligonucleotide probes as disclosed in U.S. Pat. No. 5,652,096 to G. D. Cimino.

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

In one embodiment of the present invention, it is contemplated that primers will be used for the amplification of nucleic acid sequences. Examples of such primers are SEQ ID NOS: 1, 2, 3 and 4 (see, Example 1). The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is usefully single stranded for maximum efficiency in amplification, but may alternatively be double stranded. If double stranded, the primer is first treated to separate its strands before being used to prepare extension products. Usefully, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

In one embodiment of the present invention, it is contemplated that cells and tissues will be identified for expressing the Oct-4 gene via PCR amplification. The term “polymerase chain reaction” (“PCR”) refers to the method of K. B. Mullis, U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

One embodiment of the present invention contemplates reverse-transcription of Oct-4 and connexin 43 or other connexin mRNA. The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “in operable combination,” “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

One embodiment of the present invention, contemplates the expression of Oct-4 and Connexin (for example, Connexin 43) genes. In one embodiment of the present invention, it is contemplated that the genes of the present invention may comprise promoters, regulator elements and enhancer elements. Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987). In the present invention, it is contemplated that, for example, the Oct-4 gene or Connexin 43 gene may be joined to promoter specific for muscle tissues of skeletal tissues. Examples of such promoters include, but are not limited to, the ankyrin 1 muscle promoter, the desmin gene promoter, the actin promoter and the myosin promoter. Additionally, it is contemplated that the Oct-4 and connexin 43 genes may be joined to a constitutive promoter or an inducible promoter (both defined below) or to a promoter specific for other cell or tissue types (defined below) (e.g., promoters specific for beast tissue, liver tissue or kidney tissue).

The terms “promoter element,” “promoter,” or “promoter sequence” refer to a DNA sequence that is located at the 5′ end (i.e., precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer, and is typically between about 500 bp and about 4 kb in length, and may be about 1 kb to about 1.5 kb in length.

Promoters may be tissue specific or cell specific. Examples of promoters specific for muscle tissues are given above. The term “tissue specific” as it applies to a promoter refers to a promoter that is capable of directing selective expression of a nucleotide sequence of interest to a specific type of tissue (e.g., muscle) in the relative absence of expression of the same nucleotide sequence of interest in a different type of tissue (e.g., bone). Tissue specificity of a promoter may be evaluated by, for example, operably linking a reporter gene to the promoter sequence to generate a reporter construct, introducing the reporter construct into the genome of an organism such that the reporter construct is integrated into every tissue of the resulting transgenic organism, and detecting the expression of the reporter gene (e.g., detecting mRNA, protein, or the activity of a protein encoded by the reporter gene) in different tissues of the transgenic organism. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or inducible. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, an “inducible” promoter is one that is capable of directing a level of transcription of an operably linked nucleic acid sequence in the presence of a stimulus (e.g., heat shock, chemicals, light, etc.) that is different from the level of transcription of the operably linked nucleic acid sequence in the absence of the stimulus.

The term “regulatory element” refers to a genetic element that controls some aspect of the expression of nucleic acid sequence(s). For example, a promoter is a regulatory element that facilitates the initiation of transcription of an operably linked coding region. Other regulatory elements are splicing signals, polyadenylation signals, termination signals, etc.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

The term “naturally linked” or “naturally located” when used in reference to the relative positions of nucleic acid sequences means that the nucleic acid sequences exist in nature in the relative positions.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8). An example of a commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The term “vector” refers to nucleic acid molecules that transfer DNA segment(s) from one cell to another. The term “vehicle” is sometimes used interchangeably with “vector.” In one embodiment, vectors comprising the sequences and portions of sequences of the present invention are contemplated.

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

In one embodiment of the present invention, it is contemplated that the sequences of the present invention (and portions thereof) may be used in transfection protocols. The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973), has been modified by several groups to optimize conditions for particular types of cells. The art is well aware of these numerous modifications.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “transgene” refers to a foreign gene (e.g., Oct-4 and Connexin 43) that is placed into an organism by the process of transfection. The term “foreign gene” refers to any nucleic acid (e.g., gene sequence) that is introduced into the genome of an organism by experimental manipulations and may include gene sequences found in that organism so long as the introduced gene does not reside in the same location as does the naturally-occurring gene.

The term “transgenic” when used in reference to a host cell or an organism refers to a host cell or an organism that contains at least one heterologous or foreign gene in the host cell or in one or more of cells of the organism.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal. In the present invention, it is contemplated that host cells are, for example, myoblasts, and myocytes.

The terms “transformants,” “transformed cells” or “transformed” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet, et al., Mol. Cell. Biol. 7:725, 1987 and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

In one embodiment, the present invention contemplates the overexpression of the Oct-4 and Connexin 43 genes. The term “overexpression” refers to the production of a gene product in transgenic organisms that exceeds levels of production in normal or non-transformed organisms. The term “cosuppression” refers to the expression of a foreign gene that has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

In one embodiment, the present invention contemplates a method of detecting Oct-4 and connexin 43 expression, comprising: a) providing nucleic acid samples; and b) assay said samples under conditions such that Oct-4 and connexin 43 expression are identified by, for example, Southern blotting, Northern blotting and nucleic acid sequencing. The terms “Southern blot analysis” and “Southern blot” and “Southern” refer to the analysis of DNA on agarose or acrylamide gels in which DNA is separated or fragmented according to size followed by transfer of the DNA from the gel to a solid support, such as nitrocellulose or a nylon membrane. The immobilized DNA is then exposed to a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al. (1989) Molecular Cloning: A Laboratory Manual, Cold Spring Harbor Press, NY, pp 9.31-9.58). Detection may be performed via Southern blotting. This may be performed by testing for the hybridization of a complementary test sequence (i.e., a probe for Oct-4 or Connexin 43) to the subject DNA.

The term “Northern blot analysis” and “Northern blot” and “Northern” refer to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al. (1989) supra, pp 7.39-7.52). Detection may be performed via Northern blotting. This may be performed by testing for the hybridization of a complementary test sequence (i.e., a probe for Oct-4 or Connexin 43) to the subject RNA.

In one embodiment, the present invention contemplates a method of identifying adult stem cells and metastatic cancer cells comprising: a) providing nucleic acid samples from a culture, tissue or patient; and b) assaying said samples under conditions such that cells expressing Oct-4 and/or connexin 43 are identified by, for example, Western blotting and peptide sequencing. The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies. Detection may be performed via Western blotting. This may be performed by testing for the recognition of a probe (i.e., an antibody for Oct-4 or connexin 43) to the subject peptides.

The term “antigenic determinant” refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

In one embodiment, the present invention contemplates isolated transcripts. The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

In one embodiment, the present invention contemplates purified nucleic acid and amino acid sequences. The term “purified” refers to molecules, either nucleic or amino acid sequences, that are removed from their natural environment, isolated or separated. An “isolated nucleic acid sequence” may therefore be a purified nucleic acid sequence. “Substantially purified” molecules are at least 60% free, usefully at least 75% free, and more usefully at least 90% free from other components with which they are naturally associated. As used herein, the term “purified” or “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

One embodiment of the present invention contemplates that nucleic acids, peptides, vectors, antibodies, etc, of the present invention may comprise part of a composition. The term “composition comprising” a given polynucleotide sequence or polypeptide refers broadly to any composition containing the given polynucleotide sequence or polypeptide. The composition may comprise an aqueous solution. Compositions comprising polynucleotide sequences encoding Oct-4 or fragments thereof may be employed as hybridization probes. In this case, the Oct-4 encoding polynucleotide sequences are typically employed in an aqueous solution containing salts (e.g., NaCl), detergents (e.g., SDS), and other components (e.g., Denhardt's solution, dry milk, salmon sperm DNA, etc.).

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “response,” when used in reference to an assay, refers to the generation of a detectable signal (e.g., accumulation of reporter protein, increase in ion concentration, accumulation of a detectable chemical product).

The term “sample” is used in its broadest sense. In one sense it can refer to a cell or tissue. In another sense, it is meant to include a specimen or culture, obtained from any source and encompass fluids, solids and tissues. Environmental samples include environmental material such as surface matter, soil, water, and industrial samples. These examples are not to be construed as limiting the sample types applicable to the present invention.

“Immunohistochemistry” shall be defined as, for example, the histochemical localization of immunoreactive substances using labelled antibodies as reagents.

4.3 Adult Stem Cell Markers

In certain aspects, the present invention relates to methods to screen for adult human stem cells and metastatic cancer cells. Other aspects of the present invention also relate to the screening of compounds and methods that, for example, may alter or reduce the migration of metastatic cancer cells (metastatic cells) or kill metastatic cells. 4.3.1 Oct-4 and Connexin Polynucleotides

The nucleotide sequences of the present invention may be engineered in order to alter Oct-4 (SEQ ID NO: 5) and Connexin 43 (SEQ ID NO: 6) coding sequence for a variety of reasons, including but not limited to, alterations which modify the cloning, processing and/or expression of the gene product. For example, mutations may be introduced using techniques that are well known in the art (e.g., site-directed mutagenesis to insert new restriction sites, to alter glycosylation patterns, to change codon usefulness, etc.).

The polynucleotide sequence of Oct-4 (SEQ ID NO: 5) and Connexin 43 (SEQ ID NO: 6) may be extended utilizing the nucleotide sequences by various methods known in the art to detect upstream sequences such as promoters and regulatory elements. For example, it is contemplated that restriction-site polymerase chain reaction (PCR) will find use in the present invention. This is a direct method which uses universal primers to retrieve unknown sequence adjacent to a known locus (Gobinda et al., PCR Methods Applic., 2:318-22 (1993). First, genomic DNA is amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

In another embodiment, inverse PCR can be used to amplify or extend sequences using divergent primers based on a known region (Triglia et al., Nucleic Acids Res., 16:8186 (1988). The primers may be designed using Oligo 4.0 (National Biosciences Inc, Plymouth Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template. In still other embodiments, walking PCR is utilized. Walking PCR is a method for targeted gene walking that permits retrieval of unknown sequence (Parker et al., Nucleic Acids Res., 19:3055-60 (1991). The PROMOTERFINDER® kit (Clontech) uses PCR, nested primers and special libraries to “walk in” genomic DNA. This process avoids the need to screen libraries and is useful in finding intron/exon junctions.

Useful non-limiting libraries for screening for full length cDNAs include mammalian libraries that have been size-selected to include larger cDNAs. Also, random primed libraries are useful, in that they will contain more sequences that contain the 5′ and upstream gene regions. A randomly primed library may be useful in case where an oligo d(T) library does not yield full-length cDNA. Genomic mammalian libraries are useful for obtaining introns and extending 5′ sequence.

In other embodiments of the present invention, variants of the disclosed sequences are provided. In some embodiments, variants result from polymorphisms or mutations, (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

It is contemplated to modify the structure of a peptide having a function (e.g., Oct-4 or Connexin function) for such purposes as, for example, increasing binding affinity of the Oct-4 for it's substrate or altering gap junction formation or GJIC activity (function). Such modified peptides are considered functional equivalents of peptides having an activity of Oct-4 or Connexin(s) as defined herein. A modified peptide can be produced in which the nucleotide sequence encoding the polypeptide has been altered, such as by substitution, deletion, or addition. In particular embodiments, these modifications do not significantly reduce the synthetic activity of the modified Oct-4 and Connexin 43. In other words, construct “X” can be evaluated in order to determine whether it is a member of the genus of modified or variant Oct-4's and Connexin 43's of the present invention as defined functionally, rather than structurally.

Moreover, as described above, variant forms of Oct-4 and Connexin 43 are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of Oct-4 and Connexin 43 disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer ed., Biochemistry, pp. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

As described in more detail below, variants may be produced by methods such as directed evolution or other techniques for producing combinatorial libraries of variants, described in more detail below. In still other embodiments of the present invention, the nucleotide sequences of the present invention may be engineered in order to alter a Oct-4 and Connexin 43 coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon usefulness, etc.).

4.3.2 Oct-4 and Connexin 43 Polypeptides

The present invention provides fragments, fusion proteins or functional equivalents of the Oct-4 and Connexin 43 proteins. The invention also provides nucleic acid sequences corresponding to Oct-4 and Connexin 43 variants, homologs, and mutants which may be used to generate recombinant DNA molecules that direct the expression of the Oct-4 and Connexin 43 variants, homologs and mutants in appropriate host cells. In some embodiments of the present invention, the polypeptide is a naturally purified product, in other embodiments it is a product of chemical synthetic procedures, and in still other embodiments it is produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue.

Due to the inherent degeneracy of the genetic code, DNA sequences other than the polynucleotide sequences that encode Oct-4 and Connexin 43 that encode substantially the same or a functionally equivalent amino acid sequence, may be used to clone and express Oct-4 and Connexin 43. In general, such polynucleotide sequences hybridize to the sequences that encode Oct-4 and Connexin 43 under conditions of high to medium stringency as described above. As will be understood by those of skill in the art, it may be advantageous to produce Oct-4-encoding and Connexin 43-encoding nucleotide sequences possessing non-naturally occurring codons. Therefore, in some useful embodiments, codons useful by a particular prokaryotic or eukaryotic host (Murray, et al., Nucl. Acids Res., 17 (1989) are selected, for example, to increase the rate of Oct-4 and Connexin 43 expression or to produce recombinant RNA transcripts having desirable properties, such as a longer half-life, than transcripts produced from naturally occurring sequence.

4.4 Production and Purification of Oct-4 and Connexin 43

The polynucleotides of the present invention may be employed for producing polypeptides by recombinant techniques. Thus, for example, the polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above (e.g., nucleotide sequences that encode Oct-4 and Connexin 43). In some embodiments of the present invention, the constructs comprise a vector, such as a plasmid or viral vector, into which a sequence of the invention has been inserted, in a forward or reverse orientation. In still other embodiments, the heterologous structural sequence (e.g., nucleotide sequences that encode Oct-4 and Connexin 43) is assembled in appropriate phase with translation initiation and termination sequences. In useful embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pWLNEO, pSV2CAT, pOG44, PXT1, pSG (Stratagene) pSVK3, pBPV, pMSG, pSVL (Pharmacia). Any other plasmid or vector may be used as long as they are replicable and viable in the host. In some embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 (1981), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, (1986). Alternatively, in some embodiments of the present invention, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989).

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

The present invention also provides methods for recovering and purifying Oct-4 and Connexin 43 from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein refolding steps can be used as necessary, in completing configuration of the mature protein. In still other embodiments of the present invention, high performance liquid chromatography (HPLC) can be employed for final purification steps.

The present invention further provides polynucleotides having the coding sequence (e.g., polynucleotides encoding the peptide sequences Oct-4 and Connexin 43) fused in frame to a marker sequence that allows for purification of the polypeptide of the present invention. A non-limiting example of a marker sequence is a hexahistidine tag which may be supplied by a vector, usefully a pQE-9 vector, which provides for purification of the polypeptide fused to the marker in the case of a bacterial host, or, for example, the marker sequence may be a hemagglutinin (HA) tag when a mammalian host (e.g., COS-7 cells) is used. The HA tag corresponds to an epitope derived from the influenza hemagglutinin protein (Wilson et al., Cell, 37:767 (1984).

4.5 Truncation Mutants, Fragments, Variants and Fusions of Oct-4 and Connexin 43

In addition, the present invention provides fragments of Oct-4 and Connexin 43. In some embodiments of the present invention, when expression of a portion of the Oct-4 and Connexin 43 protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat, et al., J. Bacteriol, 169:751-757 (1987) and Salmonella typhimurium and its in vitro activity has been demonstrated on recombinant proteins (Miller et al., Proc. Natl. Acad. Sci. USA 84:2718-1722 (1990). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP.

The present invention also provides fusion proteins incorporating all or part of Oct-4 or Connexin 43. Accordingly, in some embodiments, the coding sequences for the polypeptide can be incorporated as a part of a fusion gene including a nucleotide sequence encoding a different polypeptide. It is contemplated that this type of expression system will find use under conditions where it is desirable to produce an immunogenic fragment of a Oct-4 or Connexin 43 protein. In some embodiments of the present invention, the VP6 capsid protein of rotavirus is used as an immunologic carrier protein for portions of the Oct-4 or Connexin 43 polypeptide, either in the monomeric form or in the form of a viral particle. In other embodiments of the present invention, the nucleic acid sequences corresponding to the portion of Oct-4 or Connexin 43 against which antibodies are to be raised can be incorporated into a fusion gene construct which includes coding sequences for a late vaccinia virus structural protein to produce a set of recombinant viruses expressing fusion proteins comprising a portion of Oct-4 or a connexin as part of the virion. It has been demonstrated with the use of immunogenic fusion proteins utilizing the hepatitis B surface antigen fusion proteins that recombinant hepatitis B virions can be utilized in this role as well. Similarly, in other embodiments of the present invention, chimeric constructs coding for fusion proteins containing a portion of Oct-4 or Connexin 43 and the poliovirus capsid protein are created to enhance immunogenicity of the set of polypeptide antigens (See e.g., EP Publication No. 025949; and Evans, et al., Nature 339:385 (1989); Huang, et al., J. Virol., 62:3855 (1988); and Schlienger, et al., J. Virol., 66:2 (1992)).

In still other embodiments of the present invention, the multiple antigen peptide system for peptide-based immunization can be utilized. In this system, a desired portion of Oct-4 or Connexin 43 is obtained directly from organo-chemical synthesis of the peptide onto an oligomeric branching lysine core (see e.g., Posnett, et al., J. Biol. Chem., 263:1719 (1988); and Nardelli, et al., J. Immunol., 148:914 (1992)). In other embodiments of the present invention, antigenic determinants of the Oct-4 or connexin proteins can also be expressed and presented by bacterial cells.

In addition to utilizing fusion proteins to enhance immunogenicity, it is widely appreciated that fusion proteins can also facilitate the expression of proteins, such as the Oct-4 or Connexin 43 proteins of the present invention. Accordingly, in some embodiments of the present invention, Oct-4 or Connexin 43 can be generated as a glutathione-S-transferase (i.e., GST fusion protein). It is contemplated that such GST fusion proteins will enable easy purification of Oct-4 or Connexin 43, such as by the use of glutathione-derivatized matrices (See, e.g, Ausabel et al. (eds.), Current Protocols in Molecular Biology, John Wiley & Sons, NY (1991)). In another embodiment of the present invention, a fusion gene coding for a purification leader sequence, such as a poly-(His)/enterokinase cleavage site sequence at the N-terminus of the desired portion of Oct-4 or Connexin 43, can allow purification of the expressed Oct-4 or Connexin 43 fusion protein by affinity chromatography using a Ni²⁺ metal resin. In still another embodiment of the present invention, the purification leader sequence can then be subsequently removed by treatment with enterokinase (See e.g., Hochuli, et al., J. Chromatogr., 411:177 (1987); and Janknecht, et al., Proc. Natl. Acad. Sci. USA 88:8972).

Techniques for making fusion genes are well known. Usefully, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

Still other embodiments of the present invention provide mutant or variant forms of Oct-4 or Connexin 43 (i.e., muteins (proteins with altered amino acid sequence usually enough to alter properties)). The structure of a peptide having an activity of Oct-4 or Connexin 43 can be modified for such purposes as enhancing therapeutic or prophylactic efficacy, or stability (e.g., ex vivo shelf life, and/or resistance to proteolytic degradation in vivo). Such modified peptides are considered functional equivalents of peptides having an activity of the subject Oct-4 or Connexin 43 proteins as defined herein. A modified peptide can be produced in which the amino acid sequence has been altered, such as by amino acid substitution, deletion, or addition.

Moreover, as described above, variant forms (e.g., mutants or polymorphic sequences) of the subject Oct-4 or Connexin 43 proteins are also contemplated as being equivalent to those peptides and DNA molecules that are set forth in more detail. For example, as described above, the present invention encompasses mutant and variant proteins that contain conservative or non-conservative amino acid substitutions.

This invention further contemplates a method of generating sets of combinatorial mutants of the present Oct-4 or Connexin 43 proteins, as well as truncation mutants, and is especially useful for identifying potential variant sequences (i.e., mutants or polymorphic sequences). The purpose of screening such combinatorial libraries is to generate, for example, novel Oct-4 or Connexin 43 variants that can act as either agonists or antagonists, or alternatively, possess novel activities all together.

Therefore, in some embodiments of the present invention, Oct-4 or Connexin 43 variants are engineered by the present method to promote, for example, reduced migration of metastatic cells. In other embodiments of the present invention, combinatorially-derived homologs are generated which have a selective potency relative to a naturally occurring Oct-4 or Connexin 43. Such proteins, when expressed from recombinant DNA constructs, can be used, for example, in gene therapy protocols or in the generation of transgenic animals.

Still other embodiments of the present invention provide Oct-4 or Connexin 43 variants that have intracellular half-lives dramatically different than the corresponding wild-type protein. For example, the altered protein can be rendered either more stable or less stable to proteolytic degradation or other cellular process that result in destruction of, or otherwise inactivate, Oct-4 or Connexin 43. Such variants, and the genes which encode them, can be utilized to alter the location of Oct-4 or Connexin 43 expression by modulating the half-life of the protein. For instance, a short half-life can give rise to more transient Oct-4 or Connexin 43 biological effects and, when part of an inducible expression system, can allow tighter control of Oct-4 or Connexin 43 levels within the cell. As above, such proteins, and particularly their recombinant nucleic acid constructs, can be used in gene therapy protocols.

In still other embodiments of the present invention, Oct-4 or Connexin 43 variants are generated by the combinatorial approach to act as antagonists, in that they are able to interfere with the ability of the corresponding wild-type protein to regulate cell function.

In some embodiments of the combinatorial mutagenesis approach of the present invention, the amino acid sequences for a population of Oct-4 or Connexin 43 variants or other related proteins are aligned, usefully to promote the highest homology possible. Such a population of variants can include, for example, Oct-4 or Connexin 43 homologs from one or more strain or Oct-4 or Connexin 43 variants from the same strain but which differ due to mutation. Amino acids that appear at each position of the aligned sequences are selected to create a degenerate set of combinatorial sequences.

In one embodiment of the present invention, the combinatorial Oct-4 or Connexin 43 library is produced by way of a degenerate library of genes encoding a library of polypeptides which each include at least a portion of potential Oct-4 or Connexin 43 protein sequences. For example, a mixture of synthetic oligonucleotides can be enzymatically ligated into gene sequences such that the degenerate set of potential Oct-4 or Connexin 43 sequences are expressible as individual polypeptides, or alternatively, as a set of larger fusion proteins (e.g., for phage display) containing the set of Oct-4 or Connexin 43 sequences therein.

There are many ways by which the library of potential Oct-4 or Connexin 43 homologs and variants can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential Oct-4 or Connexin 43 sequences. The synthesis of degenerate oligonucleotides is well known in the art (See e.g., Narang, Tetrahedron Lett., 39:3 9 (1983); Itakura, et al., Recombinant DNA, in Walton (ed.), Proceedings of the 3rd Cleveland Symposium on Macromolecules, Elsevier, Amsterdam, pp 273-289 (1981); Itakura, et al., Ann. Rev. Biochem., 53:323 (1984); Itakura et al., Science 198:1056 (1984); Ike, et al., Nucl. Acid Res., 11:477 (1983)). Such techniques have been employed in the directed evolution of other proteins (See e.g., Scott, et al., Science 249:386-390 (1980); Roberts, et al., Proc. Natl. Acad. Sci. USA 89:2429-2433 (1992); Devlin, et al., Science 249: 404-406 (1990); Cwirla, et al., Proc. Natl. Acad. Sci. USA 87: 6378-6382 (1990); as well as U.S. Pat. Nos. 5,223,409, 5,198,346, and 5,096,815).

It is contemplated that the Oct-4 or Connexin 43 nucleic acids (and fragments and variants thereof) can be utilized as starting nucleic acids for directed evolution. These techniques can be utilized to develop Oct-4 or Connexin 43 variants having desirable properties such as, for example, reducing migration of metastatic cells. Such variants could then be used, e.g., for the generation of transgenic animals

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The useful number of base substitutions for targeted gene is usually between 1.5 and 5 (Moore and Arnold, Nat. Biotech., 14, 458-67 (1996); Leung, et al., Technique, 1:11-15 (1989); Eckert and Kunkel, PCR Methods Appl., 1:17-24 (1991); Caldwell and Joyce, PCR Methods Appl., 2:28-33 (1992); and Zhao and Arnold, Nuc. Acids. Res., 25:1307-08 (1997)). After mutagenesis, the resulting clones are selected for desirable activity. Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., Smith, Nature, 370:324-25 (1994); U.S. Pat. Nos. 5,837,458; 5,830,721; 5,811,238; 5,733,731). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer, Nature, 370:398-91 (1994); Stemmer, Proc. Natl. Acad. Sci. USA, 91, 10747-51 (1994); Crameri, et al., Nat. Biotech., 14:315-19 (1996); Zhang, et al., Proc. Natl. Acad. Sci. USA, 94:4504-09 (1997); and Crameri, et al., Nat. Biotech., 15:436-38 (1997)). Variants produced by directed evolution can be screened for Oct-4 or Connexin 43 activity (in vivo or in vitro) or for the affect on the production of, for example, cell migration (in vivo or in vitro).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of Oct-4 or Connexin 43 homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In another embodiment of the invention, the coding sequence of Oct-4 or Connexin 43 is synthesized, whole or in part, using chemical methods well known in the art (See e.g., Caruthers, et al., Nucl. Acids Res. Symp. Ser., 7:215-233 (1980); Crea and Horn, Nucl. Acids Res., 9:2331 (1980); Matteucci and Caruthers, Tetrahedron Lett., 21:719 (1980); and Chow and Kempe, Nucl. Acids Res., 9:2807-2817 (1981)). In other embodiments of the present invention, the protein itself is produced using chemical methods to synthesize either an entire Oct-4 or Connexin 43 amino acid sequence or a portion thereof. For example, peptides can be synthesized by solid phase techniques, cleaved from the resin, and purified by preparative high performance liquid chromatography (See e.g., Creighton, Proteins Structures And Molecular Principles, W H Freeman and Co, New York N.Y. (1983)). In other embodiments of the present invention, the composition of the synthetic peptides is confirmed by amino acid analysis or sequencing (See e.g., Creighton, supra).

Direct peptide synthesis can be performed using various solid-phase techniques (Roberge, et al., Science 269:202-204 (1995)) and automated synthesis may be achieved, for example, using ABI 431A Peptide Synthesizer (Perkin Elmer) in accordance with the instructions provided by the manufacturer. Additionally, the amino acid sequence of Oct-4 or connexins, or any part thereof, may be altered during direct synthesis and/or combined using chemical methods with other sequences to produce a variant polypeptide.

4.6 Detection of Oct-4, Connexins and GJIC Activity

The present invention is not limited to a particular mechanism of action. Indeed, an understanding of the mechanism of action is not necessary to practice the present invention. Nevertheless, it is contemplated that cells expressing Oct-4 and having no detectable expression of gap junction components (e.g., connexin 43) or GJIC activity are adult stem cells or metastatic cancer cells.

Accordingly, in one embodiment, the present invention provides methods for determining whether a cell is an adult stem cell or a metastatic cancer cell by detecting the expression of Oct-4 and finding no detectable GJIC activity.

A number of methods are available for analysis of expression of genes and the detection of the expression product. Such assays fall into several categories including, but not limited to, direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

In some embodiments of the present invention, sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing, for example, the sequence, SNP or mutation of interest) is sequenced using any suitable method, including but not limited to manual sequencing using radioactive marker nucleotides or automated sequencing. The results of the sequencing are displayed using any suitable method. The sequence is examined and the presence or absence of a given gene (or gene fragment), SNP or mutation is determined.

In some embodiments of the present invention, nucleotide sequences are detected using a PCR-based assay. In some embodiments, the PCR assay comprises the use of oligonucleotide primers that hybridize to the wild type sequence and other times the assay comprises the use of oligonucleotide primers that hybridize only to the variant or wild type allele of Oct-4 or connexin 43 (e.g., to the region of polymorphism or mutation).

In some embodiments of the present invention, variant sequences and alleles are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I (Third Wave Technologies, Madison, Wis.) enzyme). DNA fragments from a sample containing a SNP or a mutation will have a different banding pattern than wild type.

In some embodiments of the present invention, variant sequences or alleles are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from allelic controls.

In useful embodiments of the present invention, variant sequences are detected using a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY (1991)). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labelled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labelled probe.

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and, then, removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

In some embodiments of the present invention, genomic profiles are generated using a assay that detects hybridization by enzymatic cleavage of specific structures (INVADER assay, Third Wave Technologies; See e.g., U.S. Pat. Nos. 5,846,717; 6,090,543; 6,001,567; 5,985,557; and 5,994,069). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected by using a fluorescent plate reader, and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

In some embodiments, a MassARRAY system (Sequenom, San Diego, Calif.) is used to detect variant sequences (See e.g., U.S. Pat. Nos. 6,043,031; 5,777,324; and 5,605,798). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

In other embodiments of the present invention, antibodies (See below for antibody production) are used to determine if a cell or tissue contains an allele encoding Oct-4 and/or connexin 43. Immunohistological techniques, for example, are well known in the art. See, for instance, “Short Protocols in Molecular Biology,” 2ed, John Wiley and Sons, 1992, which is incorporated herein by reference.

GJIC activity (communication) was measured by the methods known in the art and published in U.S. Pat. Nos. 5,650,317; 5,814,511; 6,140,119 and 6,251,931 (B1). Such techniques involve, for example, the monitoring of transmission of dyes from cell-to-cell (for example, the transfer of Lucifer Yellow dye between cells with active gap junctions, referred to as the “scrape loading/dye transfer technique) (see additionally, Faucheux, N, et al, Biomaterials 25:2501-2506 (2004), or the monitoring of electrical impulses from cell-to-cell (Garcia-Dorado, D., et al., Cardiovasc. Res. 16:386-401 (2004); and LeBeau, et al., Brain Res. Bull. 62(1);1-13 (2003). Other techniques utilize fluorescent activated cell sorting (FACS) to monitor gap junction communication (Hurtado, et al., Cell Tissue Res. Feb. 14, 2004).

4.7 Kits for Analyzing Oct-4 and Connexin 43 Expression and GJIC Activity

Aspects of the present invention also provides kits for determining whether cells test positive for Oct-4 expression and/or gap junction expression (for example, by testing for the presence or absence of connexin 43) and/or GJIC activity. In some embodiments, the kits are useful for determining whether the cells or tissues tested may be adult stem cells or are metastatic. The diagnostic kits are produced in a variety of ways. In some embodiments, the kits contain at least one reagent for specifically detecting an Oct-4 encoding nucleotide or protein. In some embodiments, the kits contain reagents for detecting if the cells or tissues express GJIC activity. In certain embodiments, the reagent is a nucleic acid that hybridizes to nucleic acids containing the SNP and that does not bind to nucleic acids that do not contain the SNP. In other embodiments, the reagents are primers for amplifying the region of DNA containing the SNP. In still other embodiments, the reagents are antibodies that usefully bind either the Oct-4 or connexin 43 proteins. In some embodiments, the kit contains instructions for determining whether the cells or tissue are or contain adult stem cells or metastatic cells. In some embodiments, the kits include ancillary reagents such as buffering agents, nucleic acid stabilizing reagents, protein stabilizing reagents, and signal producing systems (e.g., florescence generating systems as Fret systems). The test kit may be packages in any suitable manner, typically with the elements in a single container or various containers as necessary along with a sheet of instructions for carrying out the test. In some embodiments, the kits also usefully include a positive control sample. These kits also contain, for example, dye or other reagents for the measurement of GJIC activity. See, for example, the section below and the references incorporated therein.

4.8 Antibodies to Oct-4 and Connexin

Antibodies can be generated to allow for the detection of Oct-4 or connexin 43 protein. The antibodies may be prepared using various immunogens. In one embodiment, the immunogen is an Oct-4 peptide or a connexin 43 peptide to generate antibodies that recognize Oct-4 or connexin 43, respectively. Such antibodies include, but are not limited to polyclonal, monoclonal, chimeric, single chain, Fab fragments, and Fab expression libraries.

Various procedures known in the art may be used for the production of polyclonal antibodies directed against Oct-4 and connexin 43. For the production of antibody, various host animals can be immunized by injection with the peptide corresponding to the Oct-4 or connexin 43 epitope including but not limited to rabbits, mice, rats, sheep, goats, etc. In a useful embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward Oct-4 or connexin 43, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature 256:495-497 (1975), as well as the trioma technique, the human B-cell hybridoma technique (See, e.g., Kozbor, et al., Immunol. Today 4:72 (1983)), and the EBV-hybridoma technique to produce human monoclonal antibodies (Cole, et al., in Monoclonal Antibodies and Cancer Therapy, Alan R. Liss, Inc., pp. 77-96 (1985)).

In an additional embodiment of the invention, monoclonal antibodies are produced in germ-free animals utilizing technology such as that described in PCT/US90/02545). Furthermore, it is contemplated that human antibodies will be generated by human hybridomas (Cote et al., Proc. Natl. Acad. Sci. USA 80:2026-2030 (1983) or by transforming human B cells with EBV virus in vitro (Cole et al., in Monoclonal Antibodies and Cancer Therapy, Alan R. Liss, pp. 77-96 (1985)).

In addition, it is contemplated that techniques described for the production of single chain antibodies (U.S. Pat. No. 4,946,778) will find use in producing Oct-4 and connexin 43 specific single chain antibodies. An additional embodiment of the invention utilizes the techniques described for the construction of Fab expression libraries (Huse, et al., Science 246:1275-1281 (1989)) to allow rapid and easy identification of monoclonal Fab fragments with the desired specificity for Oct-4 or connexin 43.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. (As is well known in the art, the immunogenic peptide should be provided free of the carrier molecule used in any immunization protocol. For example, if the peptide was conjugated to KLH, it may be conjugated to BSA, or used directly, in a screening assay.)

The foregoing antibodies can be used in methods known in the art relating to the localization of Oct-4 and connexin 43 (e.g., for Western blotting), measuring levels thereof in appropriate biological samples, etc. The antibodies can be used to detect Oct-4 and connexin 43 in a biological sample from an individual. The biological sample can be a biological fluid, such as, but not limited to, blood, serum, plasma, interstitial fluid, urine, cerebrospinal fluid, and the like, containing cells.

The biological samples can then be tested directly for the presence of Oct-4 and connexin 43 using an appropriate strategy (e.g., ELISA or radioimmunoassay) and format (e.g., microwells, dipstick (e.g., as described in International Patent Publication WO 93/03367), etc. Alternatively, proteins in the sample can be size separated (e.g., by polyacrylamide gel electrophoresis (PAGE), in the presence or not of sodium dodecyl sulfate (SDS), and the presence of Oct-4 and connexin 43 detected by immunoblotting (Western blotting). Immunoblotting techniques are generally more effective with antibodies generated against a peptide corresponding to an epitope of a protein, and hence, are particularly suited to the present invention.

Another method uses antibodies as agents to alter signal transduction. Specific antibodies that bind to the effective domains of Oct-4 and connexin 43 or other proteins involved in intracellular signalling can be used to inhibit the interaction between the various proteins and their interaction with other ligands. Antibodies that bind to the complex can also be used therapeutically to inhibit interactions of the protein complex in the signal transduction pathways leading to the various physiological and cellular effects of Oct-4 and connexin 43 function. Such antibodies can also be used diagnostically to measure abnormal expression of Oct-4 and connexin 43, or the aberrant formation of protein complexes, which may be indicative of a disease state.

4.9 Gene Therapy Using Oct-4 and Connexins

The present invention also provides methods and compositions suitable for gene therapy to alter Oct-4 and connexin 43 expression, production, or function. As described above, the present invention provides Oct-4 and connexin 43 genes and provides methods of obtaining these and similar genes from other species. Thus, the methods described below are generally applicable across many species although the useful species is human. In some embodiments, the gene therapy is performed by providing a subject with an oligonucleotide expressing Oct-4 or, in the case of connexin 43, with antisense technology. Subjects in need of such therapy are identified by the methods described above. Accordingly, subjects could be treated after birth or, in a useful embodiment, subjects are the product of transgenic engineering wherein the desired gene is incorporated into the genome of the subject before fertilization of the oocyte (see, section VI, below).

Viral vectors commonly used for in vivo or ex vivo targeting and therapy procedures are DNA-based vectors and retroviral vectors. Methods for constructing and using viral vectors are known in the art (See, e.g., Miller and Rosman, BioTech., 7:980-990, 1992). Usefully, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to a useful (for replication) region. Such techniques may be performed in vitro (i.e., on the isolated DNA) or in situ, using the techniques of genetic manipulation or by treatment with mutagenic agents.

The replication defective virus may retain the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Defective viruses, that entirely or almost entirely lack viral genes, are useful, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt, et al., Mol. Cell. Neurosci., 2:320-330 (1991)), defective herpes virus vector lacking a glycoprotein L gene (See e.g., Patent Publication RD 371005 A), or other defective herpes virus vectors (See e.g., WO 94/21807; and WO 92/05263); an attenuated adenovirus vector, such as the vector described by Stratford-Perricaudet, et al. (J. Clin. Invest., 90:626-630 (1992); See also, La Salle et al., Science 259:988-990 (1993)); and a defective adeno-associated virus vector (Samulski et al., J. Virol., 61:3096-3101 (1987); Samulski, et al., J. Virol., 63:3822-3828 (1989); and Lebkowski, et al., Mol. Cell. Biol., 8:3988-3996 (1988)).

For in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin-12 (IL-12), interferon-gamma (IFN-â), or anti-CD4 antibody, can be administered to block humoral or cellular immune responses to the viral vectors. In addition, it is advantageous to employ a viral vector that is engineered to express a minimal number of antigens.

In one embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, use is given, within the scope of the present invention, to type 2 or type 5 human adenoviruses (Ad 2 or Ad 5), or adenoviruses of animal origin (See e.g., WO94/26914). Those adenoviruses of animal origin that can be used within the scope of the present invention include adenoviruses of canine, bovine, murine (e.g., Mav1, Beard, et al., Virol., 75-81 (1990)), ovine, porcine, avian, and simian (e.g., SAV) origin. The adenovirus of animal origin may be a canine adenovirus, more usefully a CAV2 adenovirus (e.g., Manhattan or A26/61 strain (ATCC VR-800)).

The replication defective adenoviral vectors of the invention may comprise the ITRs, an encapsidation sequence and the nucleic acid of interest. Still more usefully, at least the E1 region of the adenoviral vector is non-functional. The deletion in the E1 region may extend from nucleotides 455 to 3329 in the sequence of the Ad5 adenovirus (PvuII-BglII fragment) or 382 to 3446 (HinfII-Sau3A fragment). Other regions may also be modified, in particular the E3 region (e.g., WO95/02697), the E2 region (e.g., WO94/28938), the E4 region (e.g., WO94/28152, WO94/12649 and WO95/02697), or in any of the late genes L1-L5.

In a certain embodiment, the adenoviral vector has a deletion in the E1 region (Ad 1.0). Examples of E1-deleted adenoviruses are disclosed in EP 185,573, the contents of which are incorporated herein by reference. In another embodiment, the adenoviral vector has a deletion in the E1 and E4 regions (Ad 3.0). Examples of E1/E4-deleted adenoviruses are disclosed in WO95/02697 and WO96/22378. In still another embodiment, the adenoviral vector has a deletion in the E1 region into which the E4 region and the nucleic acid sequence are inserted.

The replication defective recombinant adenoviruses according to the invention can be prepared by any technique known to the person skilled in the art (See e.g., Levrero et al., Gene 101:195 (1991); EP 185 573; and Graham, EMBO J., 3:2917 (1984)). In particular, they can be prepared by homologous recombination between an adenovirus and a plasmid that carries, inter alia, the DNA sequence of interest. The homologous recombination is accomplished following co-transfection of the adenovirus and plasmid into an appropriate cell line. The cell line that is employed should usefully (i) be transformable by the elements to be used, and (ii) contain the sequences that are able to complement the part of the genome of the replication defective adenovirus, usefully in integrated form in order to avoid the risks of recombination. Examples of cell lines that may be used are the human embryonic kidney cell line 293 (Graham et al., J. Gen. Virol., 36:59 (1977)), which contains the left-hand portion of the genome of an Ad5 adenovirus (12%) integrated into its genome, and cell lines that are able to complement the E1 and E4 functions, as described in applications WO94/26914 and WO95/02697. Recombinant adenoviruses are recovered and purified using standard molecular biological techniques, which are well known to one of ordinary skill in the art.

The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two useful regions that carry the encapsidation functions: the left-hand part of the genome, that contains the rep gene involved in viral replication and expression of the viral genes; and the right-hand part of the genome, that contains the cap gene encoding the capsid proteins of the virus.

The use of vectors derived from the AAVs for transferring genes in vitro and in vivo has been described (See e.g., WO 91/18088; WO 93/09239; U.S. Pat. No. 4,797,368; U.S. Pat. No., 5,139,941; and EP 488 528). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In another embodiment, the gene can be introduced in a retroviral vector (e.g., as described in U.S. Pat. Nos. 5,399,346, 4,650,764, 4,980,289 and 5,124,263); Mann et al., Cell 33:153 (1983); Markowitz, et al., J. Virol., 62:1120 (1988); PCT/US95/14575; EP 453242; EP178220; Bernstein, et al. Genet. Eng., 7:235 (1985); McCormick, BioTechnol., 3:689 (1985); WO 95/07358; and Kuo, et al., Blood 82:845 (1993)). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are also disclosed in WO95/02697.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that may include a part of the gag gene (Bender et al., J. Virol., 61:1639 (1987)). Recombinant retroviral vectors are purified by standard techniques known to those having ordinary skill in the art.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner, et. al., Proc. Natl. Acad. Sci. USA 84:7413-7417 (1987)); See also, Mackey, et al., Proc. Natl. Acad. Sci. USA 85:8027-8031 (1988); Ulmer, et al., Science 259:1745-1748 (1993)). The use of cationic lipids may promote encapsulation of negatively charged nucleic acids, and also promote fusion with negatively charged cell membranes (Felgner, and Ringold, Science 337:387-388 (1989)). Particularly useful lipid compounds and compositions for transfer of nucleic acids are described in WO95/18863 and WO96/17823, and in U.S. Pat. No. 5,459,127.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA. plasmid. Methods for formulating and administering naked DNA to mammalian muscle tissue are disclosed in U.S. Pat. Nos. 5,580,859 and 5,589,466.

DNA vectors for gene therapy can be introduced into the desired host cells by methods known in the art, including but not limited to transfection, electroporation, microinjection, transduction, cell fusion, DEAE dextran, calcium phosphate precipitation, use of a gene gun, or use of a DNA vector transporter (See e.g., Wu et al., J. Biol. Chem., 267:963-967 (1992)]; Wu and Wu, J. Biol. Chem., 263:14621-14624 (1988); and Williams et al., Proc. Natl. Acad. Sci. USA 88:2726-2730 (1991)). Receptor-mediated DNA delivery approaches can also be used (Curiel, et al., Hum. Gene Ther., 3:147-154 (1992); and Wu and Wu, J. Biol. Chem., 262:4429-4432 (1987)).

4.10 Transgenic Animals

The present invention contemplates the generation of transgenic animals comprising an exogenous Oct-4 and/or connexin 43 gene or alleles, homologs, mutants, or variants thereof. In useful embodiments, the transgenic animal displays an altered phenotype as compared to non-transgenic animals (for example, a reduced incidence of metastatic tumors). In some embodiments, the altered phenotype is the overexpression of mRNA for the Oct-4 or Connexin 43 gene as compared to unaltered levels of expression. In other embodiments, the altered phenotype is the decreased expression of mRNA for the Oct-4 or Connexin 43 gene as compared to unaltered levels of expression. Methods for analyzing the presence or absence of such phenotypes include Northern blotting, mRNA protection assays and RT-PCR. In other embodiments, the transgenic animals have a knock out mutation of the Oct-4 or Connexin 43 gene. Still, transfected cells are contemplated in embodiments of the present invention.).

The transgenic animals of the present invention find use in, for example, drug screens. In some embodiments, the transgenic animals (e.g., animals displaying increased Oct-4 expression) are treated with drugs or diets for the inhibition of metastasis. In other embodiments, test compounds (e.g., a drug that is suspected of being useful to decrease metastasis) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster, et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260-1264 (1976)). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. (1986)). The viral vector system used to introduce the transgene is typically a replication-defective retrovirus carrying the transgene (D. Jahner, et al., Proc. Natl. Acad Sci. USA 82:6927-693 (1985)). Transfection is easily and efficiently obtained by culturing the blastomeres on a monolayer of virus-producing cells (Van der Putten, supra; Stewart, et al., EMBO J., 6:383-388 (1987)). Alternatively, infection can be performed at a later stage. Virus or virus-producing cells can be injected into the blastocoele (D. Jahner, et al., Nature 298:623-628 (1982)). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner, et al., supra (1982)). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 (1990), and Haskell and Bowen, Mol. Reprod. Dev., 40:386 (1995)).

In other embodiments, the transgene is introduced into embryonic stem cells and the transfected stem cells are utilized to form an embryo. ES cells are obtained by culturing pre-implantation embryos in vitro under appropriate conditions (Evans, et al., Nature 292:154-156 (1981); Bradley, et al., Nature 309:255-258 (1984); Gossler, et al., Proc. Acad. Sci. USA 83:9065-9069 (1986); and Robertson, et al., Nature 322:445-448 (1986)). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468-1474 (1988)). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or to create deletion mutants. Methods for homologous recombination are described in U.S. Pat. No. 5,614,396, incorporated herein by reference.

4.11 Drug Screening Using Oct-4 and Connexins

The present invention provides methods and compositions for using Oct-4 and connexin 43 as targets for screening drugs that can alter, for example, the migration of cells (e.g., metastatic cells).

The present invention is not limited to any particular mechanism of action. Indeed, an understanding of the mechanism of action is not necessary to practice the present invention. Nevertheless, it is contemplated that Oct-4 is a transcription factor expressed in pluripotent stem cells such as embryonic stem cells, adult stem cells and metastatic cancer cells.

In one screening method, the two-hybrid system is used to screen for compounds (e.g., drug) capable of altering (e.g., inhibiting) Oct-4 function(s) (e.g., cell migration) in vitro or in vivo. In one embodiment, a GAL4 binding site, linked to a reporter gene such as lacZ, is contacted in the presence and absence of a candidate compound with a GAL4 binding domain linked to an Oct-4 fragment and a GAL4 transactivation domain II linked to a FoxD3 or Sox-2 fragment, for example. Expression of the reporter gene is monitored and a decrease in the expression is an indication that the candidate compound inhibits the interaction of Oct-4 with FoxD3 or Sox-2. Alternately, the effect of candidate compounds on the interaction of Oct-4 with other proteins (e.g., proteins known to interact directly or indirectly with FoxD3 or Sox-2) can be tested in a similar manner.

In another screening method, candidate compounds are evaluated for their ability to alter Oct-4 binding or activity by contacting Oct-2, FoxD3 (or Sox-2), FoxD3- (or Sox-2)-associated proteins, or fragments thereof, with the candidate compound and determining binding of the candidate compound to the peptide. The protein or protein fragments is/are immobilized using methods known in the art such as binding a GST-Oct-4 fusion protein to a polymeric bead containing glutathione. A chimeric gene encoding a GST fusion protein is constructed by fusing DNA encoding the polypeptide or polypeptide fragment of interest to the DNA encoding the carboxyl terminus of GST (See e.g., Smith et al., Gene 67:31 (1988)). The fusion construct is then transformed into a suitable expression system (e.g., E. coli XA90) in which the expression of the GST fusion protein can be induced with isopropyl-beta-D-thiogalactopyranoside (IPTG). Induction with IPTG should yield the fusion protein as a major constituent of soluble, cellular proteins. The fusion proteins can be purified by methods known to those skilled in the art, including purification by glutathione affinity chromatography. Binding of the candidate compound to the proteins or protein fragments is correlated with the ability of the compound to disrupt, for example, transcription and thus regulate Oct-4 physiological effects (e.g., transcription DNA of Oct-4 regulated proteins).

In another screening method, one of the components of the Oct-4 transcription factor system, such Oct-4 or a fragment of Oct-4, is immobilized. Polypeptides can be immobilized using methods known in the art, such as adsorption onto a plastic microtiter plate or specific binding of a GST-fusion protein to a polymeric bead containing glutathione. For example, GST-Oct-4 is bound to glutathione-Sepharose beads. The immobilized peptide is then contacted with another peptide with which it is capable of binding in the presence and absence of a candidate compound. Unbound peptide is then removed and the complex solubilized and analyzed to determine the amount of bound labeled peptide. A decrease in binding is an indication that the candidate compound inhibits the interaction of Oct-4 with the other peptide. A variation of this method allows for the screening of compounds that are capable of disrupting a previously-formed protein/protein complex. For example, in some embodiments a complex comprising Oct-4 or an Oct-4 fragment bound to another peptide is immobilized as described above and contacted with a candidate compound. The dissolution of the complex by the candidate compound correlates with the ability of the compound to disrupt or inhibit the interaction between Oct-4 and the other peptide.

Another technique for drug screening provides high throughput screening for compounds having suitable binding affinity to Oct-4 peptides and is described in detail in WO 84/03564, incorporated herein by reference. Briefly, large numbers of different small peptide test compounds are synthesized on a solid substrate, such as plastic pins or some other surface. The peptide test compounds are then reacted with Oct-4 peptides and washed. Bound Oct-4 peptides are then detected by methods well known in the art.

Another technique uses Oct-4 antibodies, generated as discussed above. Such antibodies capable of specifically binding to Oct-4 peptides compete with a test compound for binding to Oct-4. In this manner, the antibodies can be used to detect the presence of any peptide that shares one or more antigenic determinants of the Oct-4 peptide.

In some embodiments of the present invention, compounds are screened for their ability to inhibit the binding of pathogen components (e.g., including, but not limited to, bacterial cell surface proteins; fungi proteins, parasite proteins, and virus proteins) to Oct-4. Any suitable screening assay may be utilized, including, but not limited to, those described herein.

The present invention contemplates many other means of screening compounds. The examples provided above are presented merely to illustrate a range of techniques available. One of ordinary skill in the art will appreciate that many other screening methods can be used.

In particular, the present invention contemplates the use of cell lines transfected with Oct-4 and/or connexin 43 and variants or mutants thereof for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors. In other embodiments, the cells can be used in reporter gene assays that monitor cellular responses at the transcription/translation level. In still further embodiments, the cells can be used in cell proliferation assays to monitor the overall growth/no growth response of cells to external stimuli.

In second messenger assays, the host cells are usefully transfected as described above with vectors encoding Oct-4 or connexin 43 or variants or mutants thereof. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; see Denyer, et al., Drug Discov. Today 3:323-32 (1998); and Gonzales, et al., Drug Discov. Today 4:431-39 (1999)). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75-80 (1996)), and plate-reading systems. In some useful embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product. As described above, it is contemplated that Oct-4 interacts, for example, with FoxD3 and/or Sox-2. Therefore, in some embodiments, the reporter gene construct comprises the 5′ regulatory region (e.g., promoters and/or enhancers) of a protein whose expression is controlled by FoxD3 and/or Sox-2 in operable association with a reporter gene (see, Inohara, et al., J. Biol. Chem. 275:27823-31 (2000) for a description of the luciferase reporter construct pBVIx-Luc). Examples of reporter genes finding use in the present invention include, but are not limited to, chloramphenicol transferase, alkaline phosphatase, firefly and bacterial luciferases, beta-galactosidase, beta-lactamase, and green fluorescent protein. The production of these proteins, with the exception of green fluorescent protein, is detected through the use of chemiluminescent, colorimetric, or bioluminescent products of specific substrates (e.g., X-gal and luciferin). Comparisons between compounds of known and unknown activities may be conducted as described above.

4.12 Pharmaceutical Compositions

The present invention further provides pharmaceutical compositions which may comprise all or portions of Oct-4 and/or connexin 43 polynucleotide sequences, Oct-4 and/or connexin 43 polypeptides, inhibitors or antagonists of Oct-4 and/or connexin 43 bioactivity, including antibodies, alone or in combination with at least one other agent, such as a stabilizing compound, and may be administered in any sterile, biocompatible pharmaceutical carrier, including, but not limited to, saline, buffered saline, dextrose, and water.

Peptides can be administered to the subject intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one subject depends upon many factors, including the subject's size, body surface area, age, the particular compound to be administered, sex, time and route of administration, general health, and interaction with other drugs being concurrently administered.

Accordingly, in some embodiments of the present invention, Oct-4 and/or connexin 43 nucleotide and Oct-4 and/or connexin 43 amino acid sequences can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, Oct-4 and/or connexin 43 polynucleotide sequences or Oct-4 and/or connexin 43 amino acid sequences may be administered alone to individuals subject to or suffering from a disease.

Depending on the condition being treated, these pharmaceutical compositions may be formulated and administered systemically or locally. Techniques for formulation and administration may be found in the latest edition of Remington's Pharmaceutical Sciences (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, usefully in physiologically compatible buffers such as Hanks' solution, Ringer's solution, or physiologically buffered saline. For tissue or cellular administration, penetrants, appropriate to the particular barrier to be permeated, are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a subject to be treated.

Pharmaceutical compositions suitable for use in the present invention include compositions wherein the active ingredients are contained in an effective amount to achieve the intended purpose. For example, an effective amount of connexin 43 may be that amount that suppresses the migration of metastatic cells. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations, which can be used orally, include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For polynucleotide or amino acid sequences of Oct-4 and/or connexin 43, conditions indicated on the label may include treatment of condition related to the migration of metastatic cells or the isolation of adult stem cells.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the useful preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, usefully, dosage can be formulated in animal models (particularly murine models) to achieve a desirable circulating concentration range that inhibits or reduces the migration of metastatic cells.

A therapeutically effective dose refers to that amount of Oct-4 and/or connexin 43 that ameliorates symptoms of the disease state (e.g., the migration of metastatic cells). Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds, which exhibit large therapeutic indices, are useful. The data obtained from these cell culture assays and additional animal studies can be used in formulating a range of dosage for human use. The dosage of such compounds lies usefully within a range of circulating concentrations that include the ED₅₀ with little or no toxicity. The dosage varies within this range depending upon the dosage form employed, sensitivity of the subject, and the route of administration.

The exact dosage is chosen by the individual physician in view of the subject to be treated. Dosage and administration are adjusted to provide sufficient levels of the active moiety or to maintain the desired effect. Additional factors which may be taken into account include the severity of the disease state; age, weight, and gender of the subject; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212). Those skilled in the art will employ different formulations for Oct-4 or connexin 43 than for the inhibitors of Oct-4 or connexin 43. Administration to the bone marrow may necessitate delivery in a manner different from intravenous injections.

4.13 Cell Migration Assays

Assays for measuring cell migration activity associated with metastatic potential are widely known in the art (see, e.g., Albini (1998) Pathol. Oncol. Res. 4: 230-41; and Albini et al. (2004) Int. J. Dev. Biol. 48: 563-71). In metastasis, tumor cells migrate from the initial tumor mass throughout the whole body. Directed tumor cell motility by chemotaxis is the final step of tumor invasion, and the inhibition of this process has been a major focus of research.

Commercial kits are available for performing such assays. For example, CHEMICON's (Temecula, Calif.) 96-well Migration Assay kit is well suited for studies of cell motility or cell invasion, in particular for the identification of anti-migratory compounds. This assay provides a rapid quantifiable fluorescent-based method by which the migratory capacity of multiple cell lines can be studied simultaneously in a single assay using the 96-well format. The CHEMICON 96-well Migration Assay Kit consists of a 96-well filter plate with a feeder tray and lid. Each filter insert has a polycarbonate membrane with 8 μm pores. The undersides of the filters are left uncoated. Invasive and migratory cells will migrate through the pores of the filter in response to a chemoattractant and cling to the bottom of the polycarbonate membrane. Migrated cells are detached, lysed and labeled with a fluorescent dye that exhibits strong fluorescence when bound to cellular nucleic acids. Sample fluorescence is measured with a fluorescence microplate reader. The excitation maximum is about 480 nm; the emission maximum is about 520 nm. The CHEMICON 96-well Migration Assay Kit is suitable for assessing the effects of pharmacological compounds on the motility of tumor cells and for assaying the migratory capacity of various different cell lines in parallel.

Another useful commercial kit for assaying cell migration is the Innocyte™ (EMD Biosciences, San Diego, Calif.) Cell Adhesion Microplate Assays, which is designed for the determination of the relative attachment of adherent cell lines to extracellular matrix proteins such as Human Fibronectin, Human Vitronectin, and Human Collagen IV. Cells are seeded onto a coated substrate, and this is followed by the determination of relative cell attachment using a fluorescent dye.

### EXAMPLES

The following examples serve to illustrate certain useful embodiments and aspects of the present invention and are not to be construed as limiting the scope thereof.

### Example 1

### Oct-4 Expression in Human Adult Breast Stem Cells During Differentiation, Immortalization and Neoplastic Transformation

Oct-4 protein expression was first examined in the adult human breast epithelial cells (HBEC) by immunohistochemistry. Type 1 and Type 2 human breast epithelial cells were prepared as previously described. The immortal SV-40-immortalized cell line M13SV1, derived from Type I normal HBECs, the weakly tumorigenic M13SV1R2, and highly tumorigenic (neu oncogene-tranduced) cell line M13SV1R2 were cultured in Type 1 MSU-1 medium. Cells from different clones were subcultured and plated in the glass chamber slides and 35 mm plates until reached the desired confluency. Cells cultured on the chamber slides were fixed and followed the immunocytochemistry protocol for checking Oct-4 expression. Cells cultured on 35 mm plates were prepared for the RNA extraction.

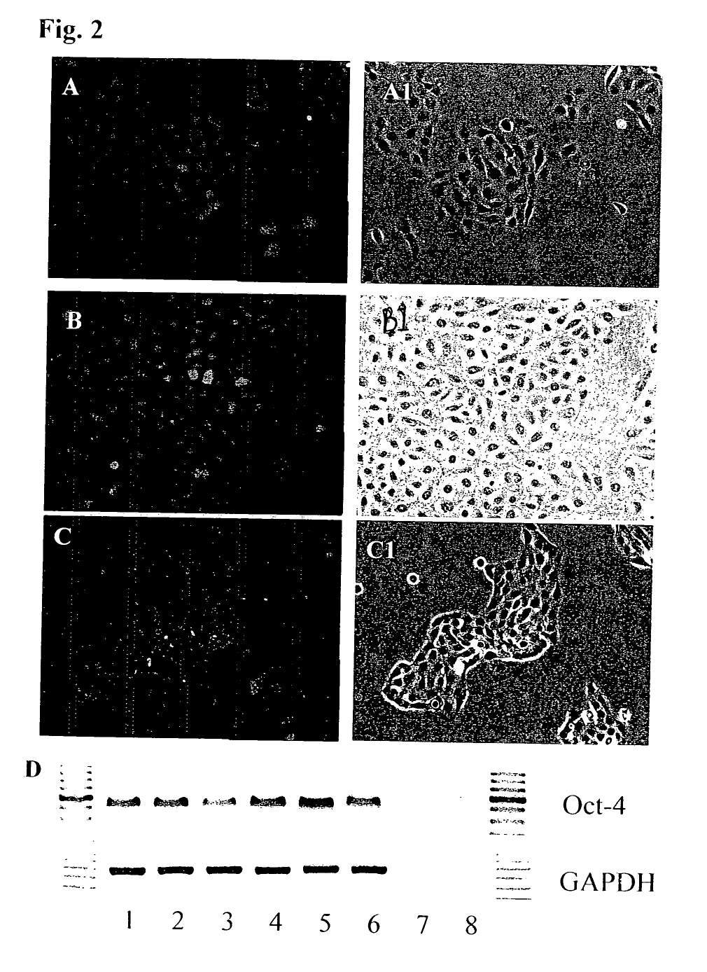

Immunohistochemistry was performed for all examples as follows. Different cell cultures were plated in glass chamber slide for a few days till it reached the desired confluency. Cells were fixed with 4% paraformaldehyde in phosphate buffer. The immunohistochemistry was performed by first blocking with 10% normal goat serum for 30 min at room temperature and incubated with primary antiserum for 2 hour at room temperature or overnight at 4° C. Cells were then rinsed off with phosphate-buffered saline (PBS) and incubated with Cyanine 3- or FITC-conjugated goat anti-rabbit or anti-mouse secondary antibodies (Jackson Immunoresearch Laboratories, West Grove, Pa.) for 1 hour at room temperature. Slides were then washed with PBS and counterstained with DAPI for 5 min before finally embedded with fluorescent mounting medium for slides. Fluorescent images were obtained using a Nikon Epi-fluorescent microscope equipped with a SPOT-RT digital camera (Diagnostic Instruments, Detroit, Mich.) interfered with Dell Pentium 4 computer installed with Spot Advanced analysis software (Diagnostic Instruments, Detroit, Mich.). The results are shown in FIGS. 2(A)-2(D).

Type I HBECs express estrogen receptor, luminal epithelial markers and have the stem cell characteristics (Kao, et al., “Two types of normal human breast epithelial cells derived from reduction mammoplasty: phenotypic characterization and response to SV40 transfection” Carcinogenesis 16:531-538, 1995) showed Oct-4 protein expression in the nucleus. The punctate staining of Oct-4 was seen homogeneously in the Type I HBECs. Type II HBECs with basal epithelial cell phenotypes as the commercially available human breast epithelial cells showed low or non-visible level of Oct-4 expression. Type I HBECs were able to differentiate into Type II cells by cyclic AMP-inducing agents or cultured in differentiation medium. Oct-4 expression in Type I cell was down-regulated when cultured in the differentiation medium. Only a few cells in the center of the colony showed the Oct-4 expression but not the majority of the cells in the colony.

The Oct-4 expression in the SV-40-immortalized cell line M13SV1 which was derived from Type I normal HBECs, the weakly tumorigenic M13SV1R2 and highly tumorigenic (the neu oncogen-transduced) cell lines M13SV1R2N1 was also examined (Kang, et al., Mol. Carcinog. 21:225-233, (1998). Oct-4 protein was all seen in high level of these three cell lines tested. High magnification of the images showed the Oct-4 protein located in the nucleus.

The presence of Oct-4 transcripts in the HBECs was determined by reverse transcription-PCR. Reverse Transcriptase Polymerase Chain Reaction was performed in each Example as follows. Total RNA was extracted from the cells with TRIZOL following the manufacturer-suggested protocols and treated with DNase I to remove contaminating DNA. Oligo(dT) primers (Integrated DNA Technologies, Iowa) were used with Superscript II reverse transcriptase (Invitrogen) for cDNA synthesis from 1 μg of total RNA following the guidelines provided by the manufacturer. PCR was conducted with PlatinumTaq Polymerase (Invitrogen) in 25 μl reaction volumes. The reaction mix consisted of 2 μl of cDNA, 10×PCR buffer, 10 mM dNTP mix, 50 mM MgCl2, 10 μM of each primer (forward and reverse), 1 unit PlatinumTaq polymerase, and milli-Q water. Glyceraldehyde 3-phosphate dehydrogenase (GAPDH) was used as an internal standard for Oct-4 RT-PCR. The two primer sequences of human Oct-4 and human GAPDH were: sense 5′-GACAACAATGAGAACCTTCAGGAGA-3′ (SEQ ID NO: 1) and antisense 5′-CTGGCGCCGGTTACAGAACCA-3′ (SEQ ID NO: 2), 5′-GTTCGACAGTCAGCCGCATC-3′ (SEQ ID NO: 3) and antisense 5′-GTGGGTGTCGCTGTTGAAGTC-3′ (SEQ ID NO: 4), respectively. The mixture was first heated at 94° C. for 3 min in a PTC-200 DNA Engine Thermal-Cycler (MJ Research, Waltham, Mass.). Amplification were performed in 35 cycles at 94° C. for 45 seconds, 55° C. for 30 seconds and 72° C. for 1 min 30 seconds followed by 72° C. for 10 minutes. The PCR products were separated on 1.5% agarose gel by electrophoresis. Digital images were captured on a KODAK gel documentation system.

The total RNA extracted from cell cultures subjected to reverse transcription-PCR using primer sets designed to amplify fragments of Oct-4 and GAPDH (positive control). Human breast stem cells showed positive Oct-4 expression. Human breast stem cells in transition into differentiated cells showing decreased but persistent Oct-4 expression. Human breast immortal, weakly tumorigenic and highly tumorigenic cell lines showed very strong Oct-4 expression. In other words, Oct-4 message was present in all the HBEC cells except Type II HBECs showed much lower expression level.

GJIC activity was measured by the methods published in U.S. Pat. Nos. 5,650,317; 5,814,511 and 6,140,119. For example, one assay measures the transfer of Lucifer Yellow dye between cells, i.e., the scrape loading/dye transfer technique. The dye is only passed form cell-to-cell if GJIC activity is present. In brief, the technique is performed as follows. Cells were grown to about 80% confluency, rinsed several times with PBS and exposed to 0.05% Lucifer Yellow dye solution (Sigma) in PBS. Several cuts were made on the monolayer of cells with a scalpel in the presence of the dye mixture to load dye into cells. The cells remained in the dye solution at room temperature for four minutes. After removing the dye solution, the cells were rinsed several times with PBS and examined using phase contrast epifluoresecent microscopy to assess the extent of dye transfer. In this example, cells expressing Oct-4 showed no or virtually no GJIC activity via the Lucifer Yellow dye transfer assay.

### Example 2

### Oct-4 Expression in Adult Human Pancreatic Stem Cells During Differentiation and Pancreatic Cancer Cells

Human pancreatic stem cells (HPC) were established as follows. Human pancreatic islets, duct and acini remnants were obtained from the JDRF Human Islet Distribution Program. Upon arrival cells were plated and cultured in RPMI-1640 medium supplemented with 8.0 mM glucose, 10% fetal bovine serum (FBS), 100 units per ml penicillin, 100 μg per ml streptomycin and 0.5 μg per ml fungizone (Amphotericin B). All cell cultures were maintained at 37° C. in a humidified atmosphere of 95% air and 5% carbon dioxide. After 24 h, the majority of islets and pancreatic cells were adhered to the culture dish surface. The culture medium was then switched to Keratinocyte Serum-Free medium (KSFM) supplemented with 5 ng/ml human recombinant epidermal growth factor (EGF), 50 μg/ml bovine pituitary extract (BPE), 2.0 mM N-acetyl cysteine (NAC) and 0.2 mM L-ascorbic acid-2-phosphate. A couple of days later, long-shaped (serpiginous (creeping; said of lesions which heal over one portion while continuing to advance at another), >90%) cells were emanating from the attached tissues. When cell cultures reached confluence they were detached with trypsin-EDTA and passed to fresh culture dishes at a density of 2.0×10⁶ cells per 100 mm dish. Early passages of cells (passage 1-3) were also cryopreserved in 80% KNC medium, 10% FBS, and 10% DMSO and stored in liquid nitrogen. Viability of cryopreserved cells was greater than 90% and there were no discernible differences in growth or morphology. For all experiments shown, cells were expanded for a minimum of three passages in KNC medium.

To induce pancreatic endocrine cell differentiation from the progenitor cells, the cells were plated and cultured in Neurobasal medium containing 1% N2 Supplement (N2 medium) with 10 mM nicotinamide (Sigma, St. Louise, Mo.), 0.1 mM exendin-4 (Bachem, Torrance, Calif.) and 5 mM LY 294002 (Calbiochem, La Jolla, Calif.) for 14 days before RNA from the cells was extracted.

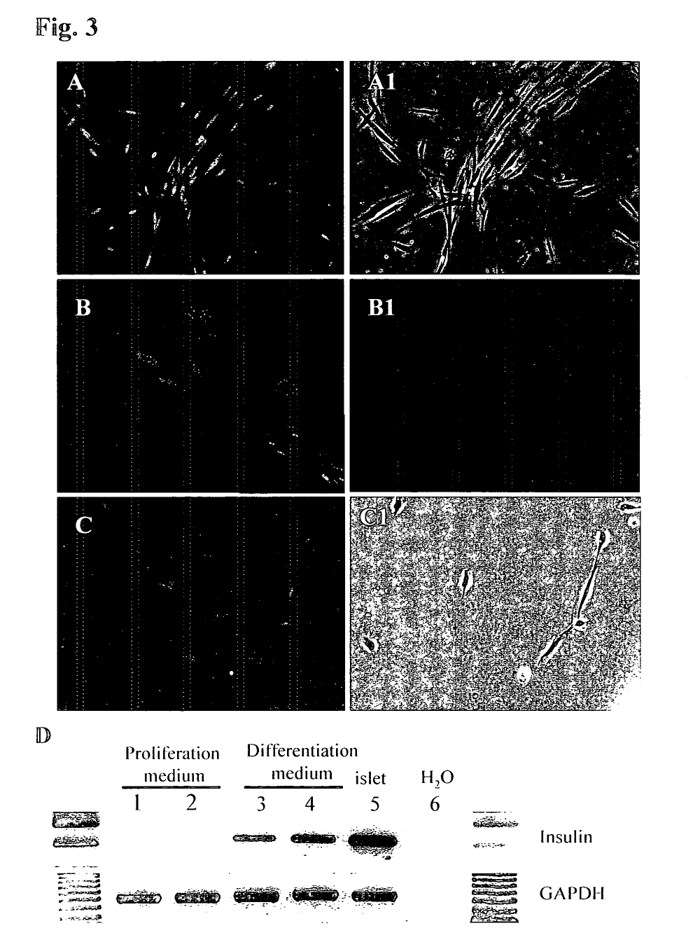

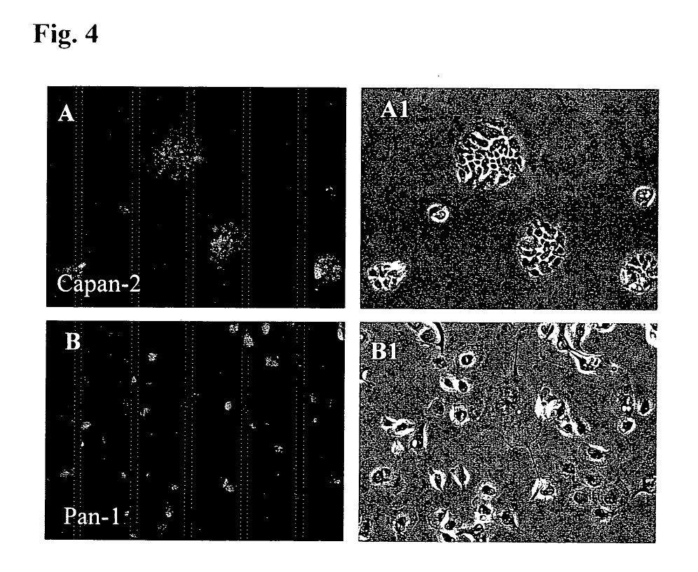

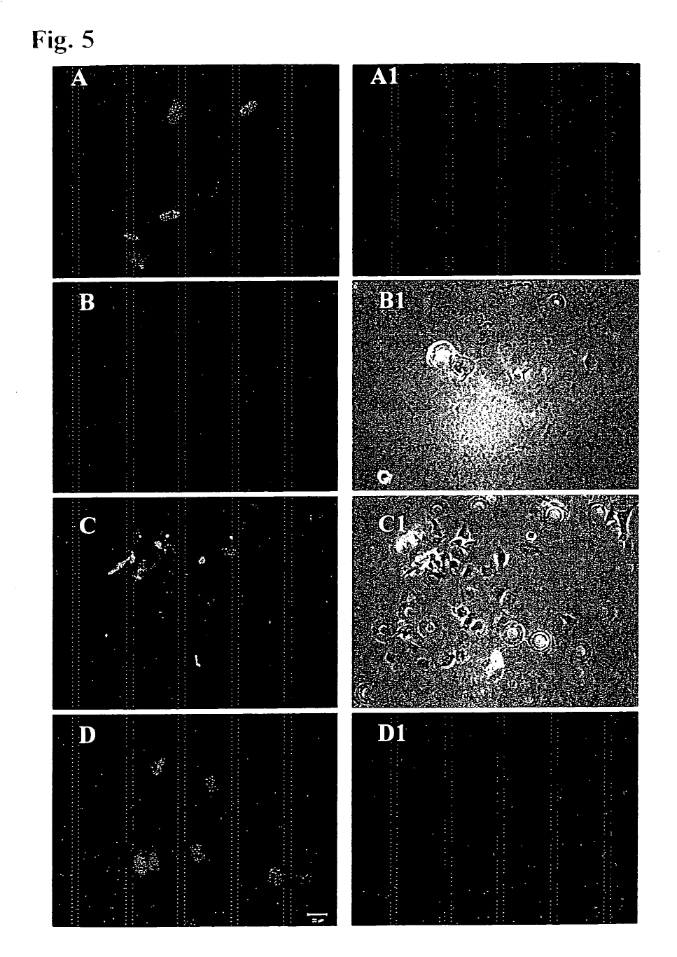

The Oct-4 expression of the human pancreatic stem cells was examined by using the immunohistochemistry. The human pancreatic stem cells were cultured in proliferation medium (KNC medium which is low calcium medium plus anti-oxidant reagents, N-acetyl-L-cysteine and ascorbic acid). Cells were fixed and double-labeled with 4′,6′-diamidino-2-phyenylindole (DAPI) for DNA (blue) and Oct-4 (red). The results are shown in FIGS. 3(A)-3(C). Oct-4 proteins showed nicely punctuated staining in the nucleus of human pancreatic stem cells. The punctate staining indicates the Oct-4 protein expression in the pancreatic stem cells.

Oct-4 protein expression was shown in human pancreatic differentiated daughter cells. Cells were fixed and double-labeled with DAPI (blue) for DNA and Oct-4 (green). Oct-4 expression was decreased in human pancreatic cells cultured in the differentiation medium (neuronal medium plus N2 supplement, LY294002 and nicotinamide). When the cells were treated in differentiation medium, Oct-4 staining was much weaker. The strategy used to stimulate endocrine cell differentiation was to culture the pancreatic stem cells in the Neurobasal medium containing 1% N2 supplement and combinations of 10 mM nicotinamide, 0.1 mM exendin-4 and 10 mM LY294002.

When the cells were cultured in the differentiation medium for two weeks, insulin message were revealed by RT-PCR. The results are shown in FIG. 3(D). Insulin mRNA expression was shown in human pancreatic stem cells and differentiated cells. Total RNA extracted from cell cultures was subjected to reverse transcription-PCR using primer sets designed to amplify fragments of insulin and GADPH (positive control). Human pancreatic stem cells cultured in the proliferation medium (KNC) showed no insulin message. Human pancreatic stem cells that were cultured in the differentiation medium showed insulin mRNA expression. Insulin expression of human islet RNA was used as a positive control. The negative control was a lane run with no template. On the other hand, it was found that Oct-4 mRNA was down-regulated when the cells were treated in the differentiation medium as shown by RT-PCR. These results indicated under this differentiation condition, pancreatic stem cells lost their stem cell marker Oct-4 and began to differentiate into insulin-positive pancreatic cells.

Oct-4 expression of human pancreatic cancer cell lines (Capan-2 (ATCC, Manassas, Va.; No. HTB-80) and Pan-1 was also examined). Capan-2 cells were grown as colonies with distinct cell boundary and uniform cell shape. Oct-4 expression was shown homogeneously punctate staining in the nucleus of Capan-2 cells. On the other hand, Pan-1 cells showed variable cell shape. Oct-4 expression was shown heterogeneously staining in the nucleus of Pan-1 cells. The presence of Oct-4 transcripts in Capan-2 and Pan-1 cells was determined by reverse transcription-PCR. Oct-4 messages were presented in both Capan-2 and Pan-1 cell lines.

GJIC activity was measured by the methods published in U.S. Pat. Nos. 5,650,317, 5,814,511, and 6,140,119. For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 3

### Oct-4 Expression in Adult Human Liver Stem Cells, Immortalized Cell and Carcinoma Cell Lines

The procedure to obtain human adult liver stem/precursor cells of clonal origin has been described (Chang, et al., 2004b; Tsai, et al., (2004), Proc. Am. Assoc. Cancer Res., 45:642). The stem cell features of these cells include 1) high proliferation potential, more than 50 cumulative population doublings (cpdl); 2) deficiency in GJIC; 3) ability of anchorage independent growth; and 4) the expression of liver “oval cell” markers, i.e. vimentin, a-fetoprotein.

Human liver stem cells were propagated and cultured in KNC medium. Oct-4 expression was examined in the human liver stem cells by using the immunohistochemistry. The results are shown in FIGS. 5(A)-5(D). Oct-4 proteins were shown clear punctate staining in the nucleus of human liver stem cells. These cells also showed vimentin (a 58 kD intermediate filament protein) and α-fetoprotein (a foetal protein found in small amounts in adults) positive by immunohistochemistry. When the cells were cultured in differentiation medium (D-medium), Oct-4 expression was diminished (see, next paragraph).

The Oct-4 expression in the SV40 immortalized liver cell line and liver tumor cell line (Mahalva cells) was also examined. Oct-4 immunostaining was shown in the immortal liver (SV40-transfected cells) and tumor lines. Oct-4 staining is not visible in the liver differentiated cells, as mentioned above. Oct-4 punctate staining was shown in both SV40 immortalized liver cell line and Mahalva cells. RT-PCR analysis also demonstrated that Oct-4 mRNA was presented in the liver stem cell, SV40 immortalized liver cell line and Mahalva cells.

GJIC activity was measured by the methods published in U.S. Pat. Nos. 5,650,317; 5,814,511 and 6,140,119. For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 4

### Oct-4 Expression in Adult Human Mesenchymal Stem Cells

The development of mesenchymal stem cells from adipose tissues used a similar method as that described for liver stem cells (Chang, et al., 2004b; Tsai, et al., (2004), Proc. Am. Assoc. Cancer Res., 45:642). Although, the derivation of mesenchymal stem cells from adipose tissues has been reported before (Zuk, et al., (2001) Tissue Eng., 7:211-228), the method is superior in having high proliferation potential to obtain larger number of cells in shorter time (32 cpdl in 51 days compared to 22 cpdl in 165 days) (Lin, et al., (2004), Stem Cell Dev., (in press)). It has been shown that these cells had high differentiation ability to become adipocytes, osteoblasts, and chondrocytes (Lin, et al., (2004), Stem Cell Dev., (in press)).

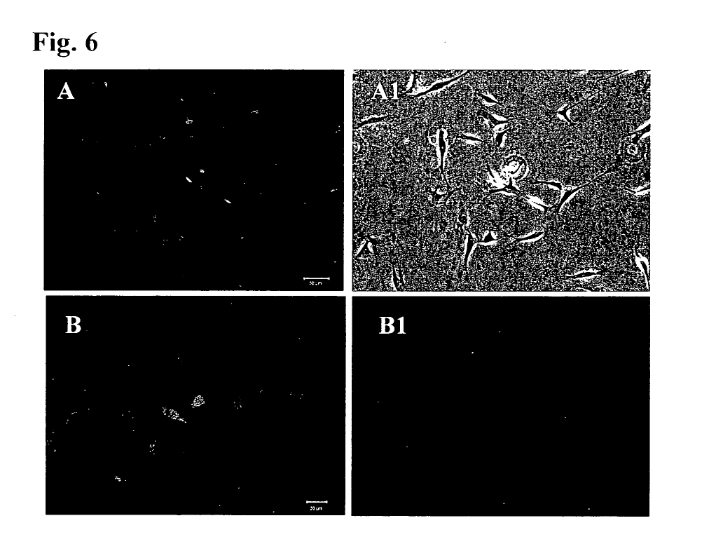

The Oct-4 expression of the human mesenchymal stem cells was examined by using the immunohistochemistry. The results are shown in FIGS. 6(A)-6(B). Oct-4 expression was shown in human mesenchymal stem cells as punctate staining in the nucleus.

GJIC activity was measured by the methods published in U.S. Pat. Nos. 5,650,317; 5,814,511 and 6,140,119. For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 5

### Oct-4 Expression in Adult Human Kidney Stem Cells

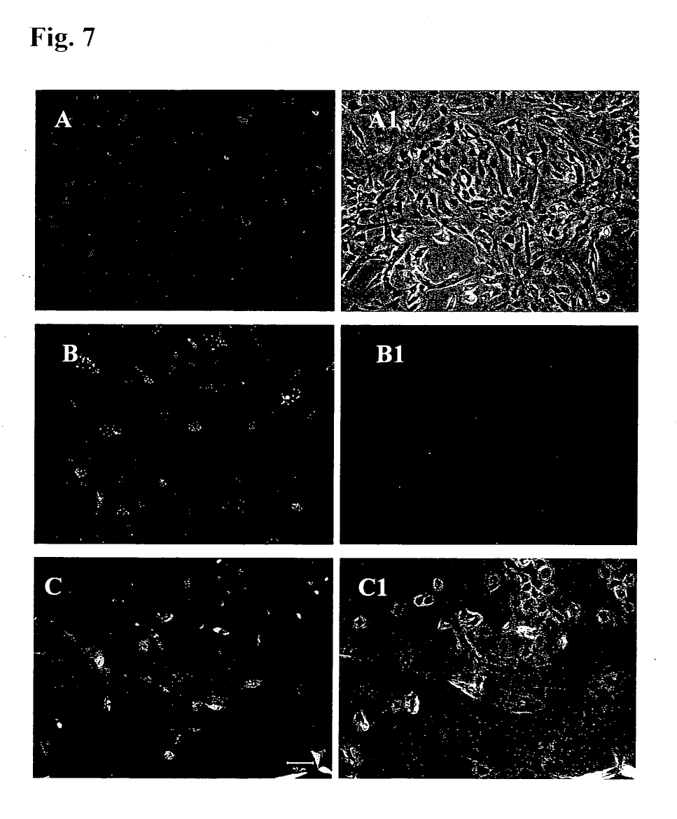

The method to develop putative human fetal kidney epithelial stem cells has been previously reported (Chang, et al., (1987), Cancer Res., 47:1634-1645). These putative kidney epithelial stem cells have been shown to be contact insensitive, i.e. the ability to form colonies on x-ray lethally irradiated human fibroblast cell mats, and to be deficient in gap junctional intercellular communication (GJIC) (Chang, et al., (1987), Cancer Res., 47:1634-1645). Oct-4 protein expression was shown by immunostaining in normal human kidney stem cells as punctate staining in the nucleus. The results are shown in FIGS. 7(A)-7(B).

GJIC activity was measured by the methods published in U.S. Pat. Nos. 5,650,317, 5,814,511, and 6,140,119. For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 6

### Oct-4 Expression in Adult Human Gastric Stem Cells

The development of gastric stem/precursor cells from adipose tissues used a similar method as that described for liver stem cells (Chang, et al., 2004b; Tsai, et al., (2004), Proc. Am. Assoc. Cancer Res., 45:642). The stem cell characters of the putative human gastric stem/precursor cells are the high differentiation ability to become various cell types (neuronal, endocrine cell-like etc.) and the readiness of these cells to become immortal spontaneously (Wu, et al., 2004).

The Oct-4 expression of the human gastric stem cells was examined by using the immunohistochemistry. The results are shown in FIG. 7(C). Oct-4 protein was shown as punctate staining in the nucleus of a heterogeneous human gastric stem cell culture.

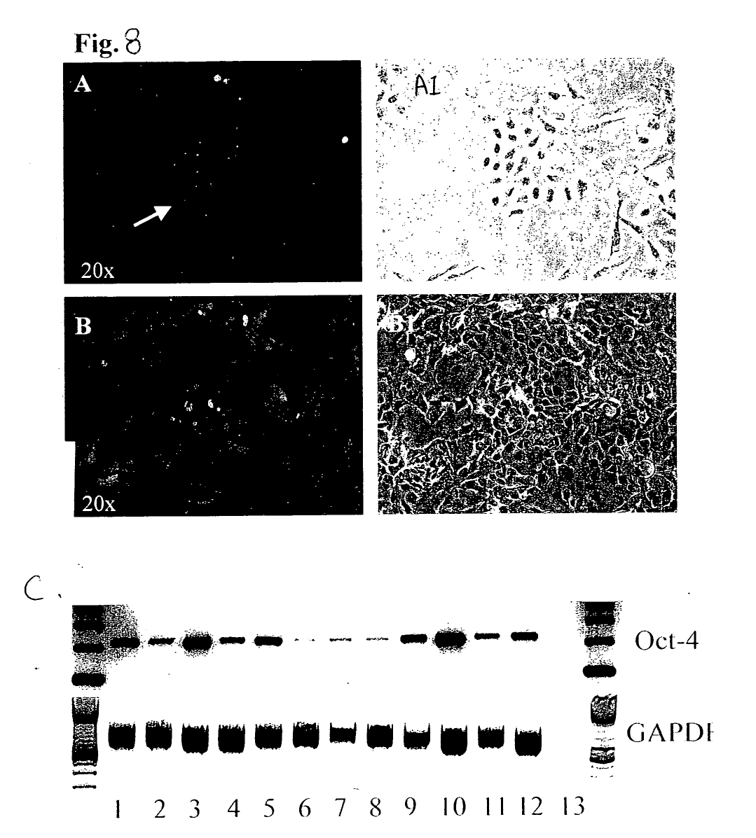

GJIC activity was measured by the methods published in U.S. Pat. Nos. 5,650,317; 5,814,511 and 6,140,119. The results are shown in FIG. 8(C). For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 7

### Oct-4 Expression in HeLa and MCF7 Cells

Oct-4 protein expression was shown in HeLa and MCF-7 transformed cell lines by immunohistolochemistry. The results are shown in FIGS. 8(A)-(B). HeLa cells showed heterogeneous expression of Oct-4 when cultured in the differentiation medium. A colony in the center showed very intense Oct-4 expression in the nucleus of the cells compared to no expression of Oct-4 of the cells on the side of the colony. Low staining of Oct-4 expression was shown in the MCF7 cells when cultured in the differentiation D medium.

The presence of oct-4 transcripts in different stem cells, immortalized cells and cancer cell lines was determined by reverse-transcription PCR. The results are shown in FIG. 8(C). Oct-4 primers and cDNA isolated from 12 different cell types resulted in PCR products in approximate size of 225 bp as expected for the human sequence.

GJIC activity was measured by the methods published in U.S. Pat. Nos. 5,650,317; 5,814,511 and 6,140,119. For example, one assay measures the transfer of Lucifer Yellow dye between cells. The dye is only passed form cell-to-cell if GJIC activity is present. In this example, cells expressing Oct-4 showed no or virtually no GJIC activity via the Lucifer Yellow dye transfer assay.

### Example 8

### Oct-4 Expression in Normal Human Skin Sections and Rat Pancreatic Tumor in vivo

The MaxArray Human Normal Tissue was purchased from Zymed to examine the Oct-4 expression in the normal human tissue. Human normal tissue sections were purchased from Zymed (San Francisco, Calif.). Zymed's MaxArray Human Normal Tissue Arrays contain 30 human samples arrayed on microscope slides. Slides were deparaffinized and rehydrated and followed the protocol of immunohistochemistry. The slides were analyzed under bright field microscope for Oct-4 staining. Rat tumor sections were provided by Dr. L. Karl Olson from Dept. of Physiology, Michigan State University. Rat tumor sections were proceeded the same protocol for Oct-4 immunohistochemistry.

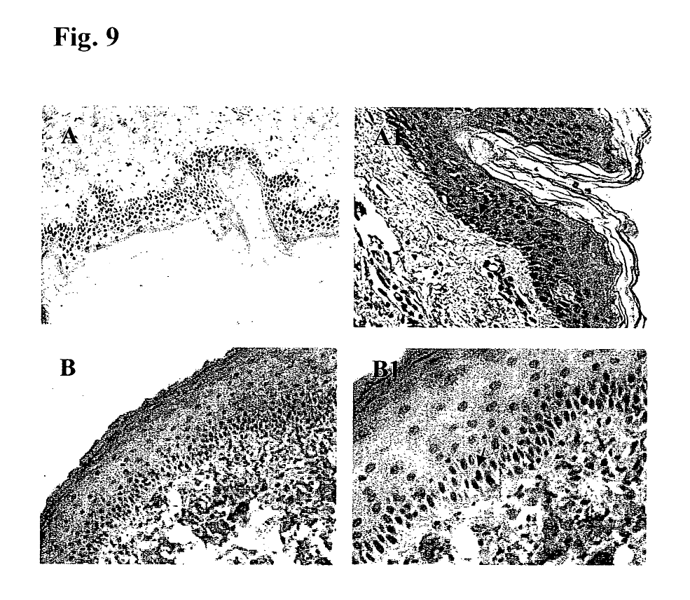

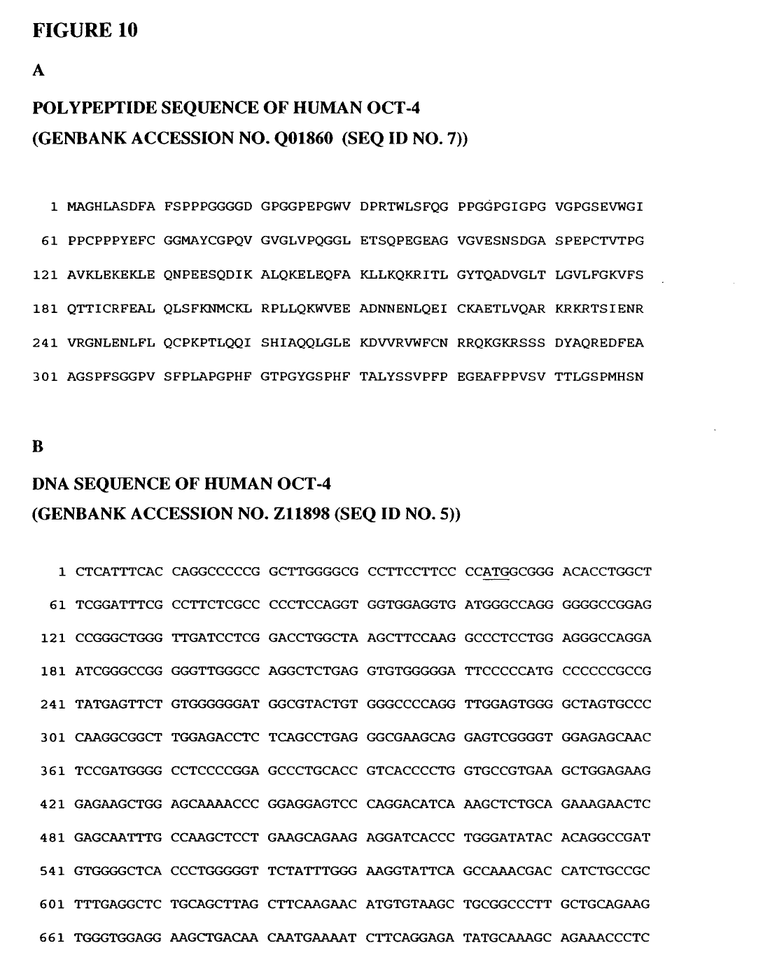

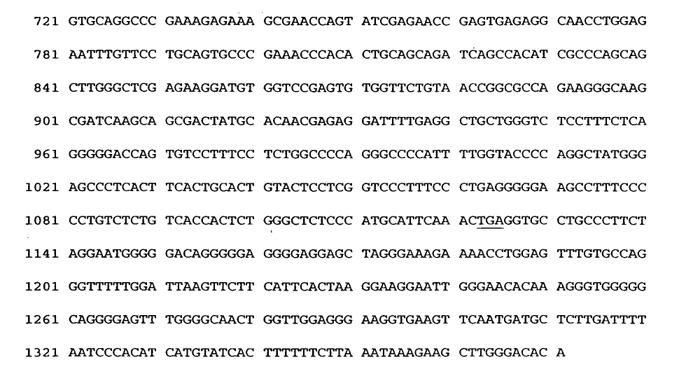

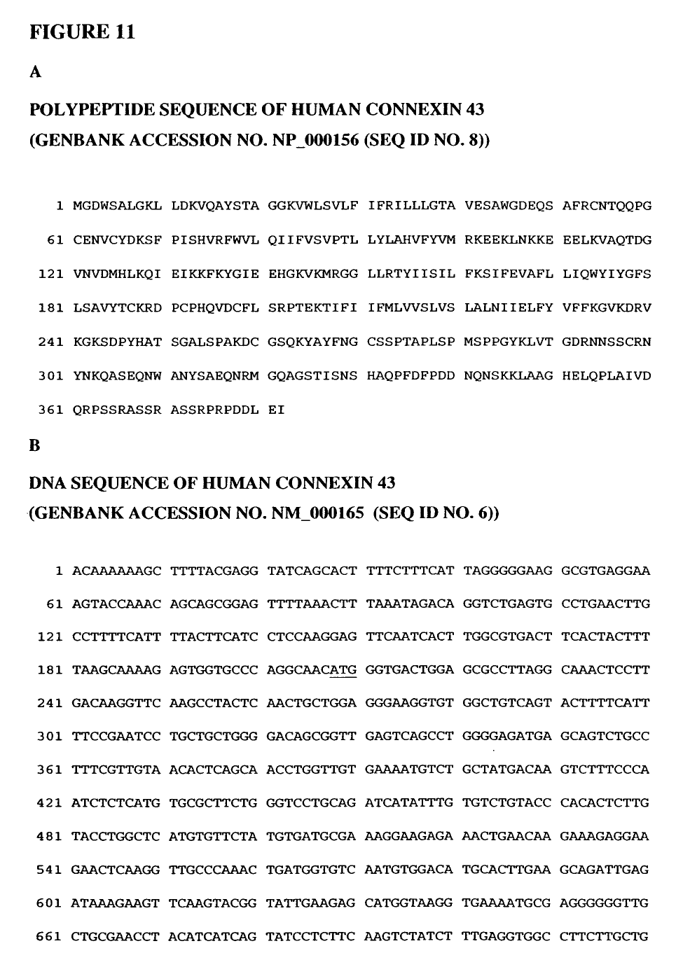

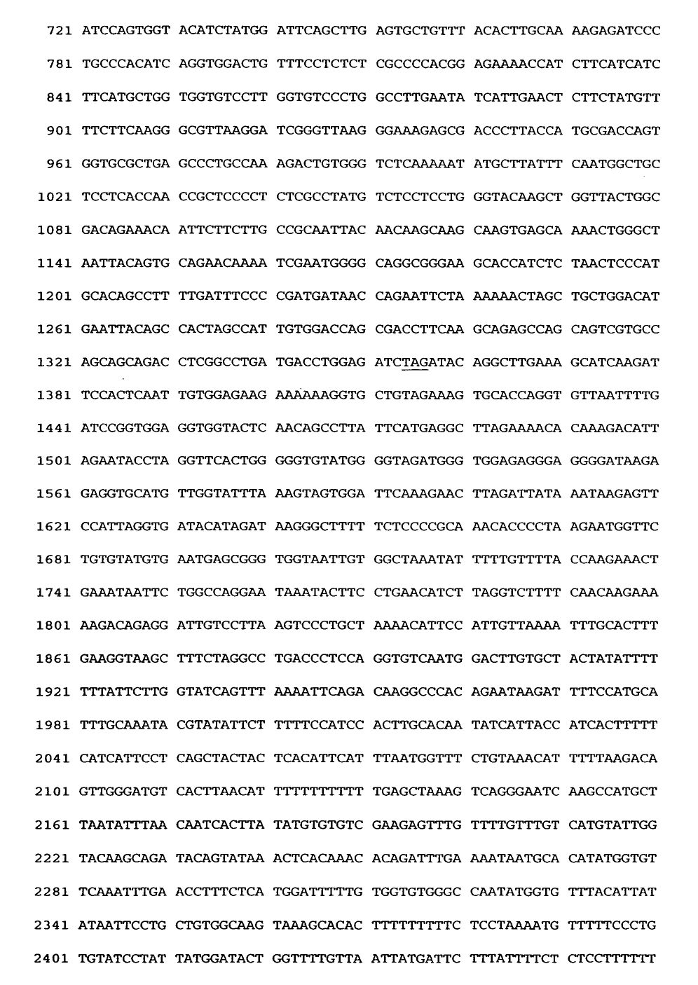

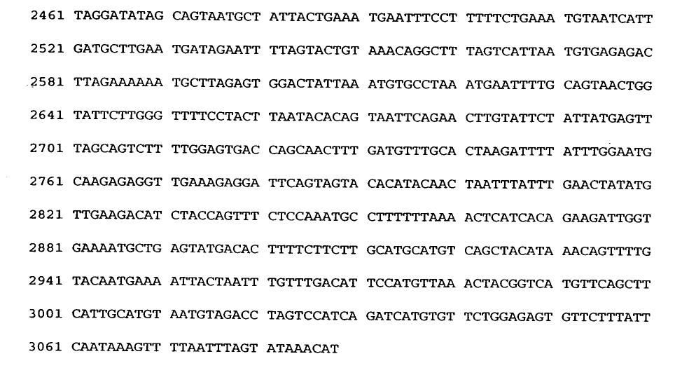

Among those 30 different normal tissue sections, the Oct-4 expression was identified in some of the cells of the skin tissue. The results are shown in FIGS. 9(A)-(B). Oct-4 protein expression was identified by the brown condensed stain. Majority of the Oct-4 stain was co-localized with blue hematoxylin stain indicated the Oct-4 protein was expressed in the nucleus. The Oct-4 positive cells were some cells scattered in the basal layer of the skin. This shows that skin stem cells were located in the basal layer of the skin. The results demonstrated that Oct-4 positive cells in the basal layer of skin are the stem cells that resided in the skin.

Additionally, Oct-4 protein expression was detected in rat pancreatic tumor sections. Rat pancreatic tumor sections were de-paraffinized and subsequently stained with Oct-4 primary antibody and avidin-HRP and finally visualized with DAB (Showing in dark brown color). Oct-4 expression was shown in the peripheral cells of the tumor tissue but not in the interior tumor cells. This suggests the existence of “cancer stem cells” and the partially-differentiated cancer cells caused by differentially micro-environmental conditions in vivo.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A method of identifying an adult mammalian stem cell in a population of adult mammalian cells comprising:
(a) contacting the adult mammalian cell population with an Oct-4 probe; and
(b) identifying cells that express Oct-4,
wherein cells that express Oct-4 are adult mammalian stem cells.

2. The method of claim 1, wherein the mammalian cells are human cells.

3. The method of claim 1, wherein the population of adult mammalian cells are from an adult mammalian tissue.

4. The method of claim 3, wherein the adult mammalian tissue is selected from the group consisting of breast, liver, pancreas, kidney, mesenchyme and gastric tissue.

5. The method of claim 4, wherein the adult mammalian tissue is from a mammal selected from the group consisting of human, dog, and rat.

6. The method of claim 1, further comprising isolating the adult mammalian stem cell that expresses Oct-4 from the population of mammalian cells.

7. The method of claim 1, further comprising detecting GJIC expression or activity.

8. The method of claim 7, wherein the absence of GJIC expression or activity in an adult mammalian stem cell that expresses Oct-4 identifies that cell as an adult mammalian stem cell.

9. The method of claim 1, further comprising obtaining a replicate population of the adult mammalian cell population.

10. The method of claim 9, wherein the replicate population is obtained by:
(a) isolating individual cells of the adult mammalian cell population; allowing the isolated individual cells of the adult mammalian cell population to divide at least once to provide two or more replicate cells; and
(b) separating the two or more replicate cells of each of the isolated individual cells so as to provide a replicate population.

11. The method of claim 10, wherein the isolated individual cells of the adult mammalian cell population are allowed to divide at least once in a low calcium cell culture medium.

12. The method of claim 10, further comprising obtaining an isolated mammalian adult stem cell from a replicate population.

13. The method of claim 12, wherein the isolated mammalian adult stem cell is obtained from the replicate of a cell that express Oct-4.

14. The method of claim 13, further comprising detecting GJIC expression or activity, wherein the absence of GJIC expression or activity in the cell that express Oct-4 further identifies the cell that expresses Oct-4, and its replicate, as adult mammalian stem cells.

15. The method of claim 13, wherein the mammalian cells are selected from the group consisting of human breast cells, human liver cells, human pancreatic cells, human kidney cells, human mesenchyme cells, and human gastric tissue cells.

16. A method of screening a population of non-embryonic mammalian cells comprising detecting the presence or absence of Oct-4 expression and the presence or absence of GJIC expression or activity in individual cells of the population.

17. The method of claim 16, wherein the presence of Oct-4 expression and the absence of GJIC expression or activity in an individual cell of the population indicates that the cell is an adult mammalian stem cell.

18. The method of claim 17, wherein the population of non-embryonic mammalian cells are adult human cells selected from the group consisting of breast cells, liver cells, pancreatic cells, kidney cells, mesenchyme cells and gastric cells.

19. The method of claim 16, wherein the presence of Oct-4 expression and the absence of GJIC expression or activity in an individual cell of the population indicates that the cell is a metastatic cancer cell.

20. A method of testing a population of mammalian cells for stem cell character or identity by providing a sample of mammalian cells; and testing the cells for the presence of Oct-4 gene expression and gap junctional intercellular communication activity, wherein the presence of Oct-4 gene expression and absence of gap junctional intercellular communication activity in a tested cell indicates that the cell is a stem cell.

21. The method of claim 20, further comprising identifying cells that test positive for Oct-4 gene expression and show an absence of gap junctional intercellular communication activity.

22. The method of claim 20, wherein the sample of mammalian cells is obtained by biopsy.

23. The method of claim 22, wherein the biopsy is a selected from the group consiting of a breast tissue biopsy, a liver tissue biopsy, a pancreatic tissue biopsy, a mesenchymal tissue biopsy, a gastric tissue biopsy, and a kidney tissue biopsy,

24. The method of claim 20, wherein the cells in the sample of cells to be tested are lysed.

25. The method of claim 20, wherein the presence of Oct-4 gene expression is tested by a method selected from the group consisting of Western blotting, Southern blotting, Northern blotting, immunohisto-chemistry, PCR, RT-PCR, HPLC, mass spectroscopy, and enzymatic detection.

26. The method of claim 20, wherein the presence of gap junctional intercellular communication activity is tested by detecting the cell-to-cell transfer of Lucifer Yellow dye.

27. The method of claim 20, wherein the presence of Oct-4 gene expression and gap junctional intercellular communication activity are each, independently of the other, measured by a method selected from the group consisting of Western blotting, Southern blotting, Northern blotting, immunohistochemistry, PCR, RT-PCR, high pressure liquid chromatography (HPLC), mass spectroscopy, DNA chip assay and enzymatic detection assay.

28. A method of detecting carcinogenic activity in a test compound comprising:
(a) providing an adult mammalian stem cell expressing Oct-4;
(b) contacting the adult mammalian stem cell expressing Oct-4 with the test compound; and
(c) allowing the adult mammalian stem cell expressing Oct-4 to grow under conditions that normally promote differentiation and loss of Oct-4 expression,
wherein the continued expression of Oct-4 under conditions that normally promote differentiation and loss of Oct-4 expression indicates that test compound possesses carcinogenic activity.

29. The method of claim 28, further comprising detecting GJIC expression or activity in the adult mammalian stem cell, wherein the absence of GJIC expression or activity under conditions that normally promote differentiation and loss of Oct-4 expression further indicates that the test compound possesses carcinogenic activity.

30. The method of claim 28, wherein the adult mammalian stem cell expressing Oct-4 is a human adult stem cell.

31. The method of claim 30, wherein the human adult stem cell is selected from the group consisting of breast stem cells, liver stem cells, pancreatic stem cells, kidney stem cells, mesenchyme stem cells and gastric stem cells.

32. A method of screening compounds for anti-cancer activity comprising:
(a) providing an adult mammalian stem cell expressing Oct-4, the adult mammalian stem cell having been exposed to a carcinogenic agent such that it continues to express Oct-4 activity under differentiating conditions;
(b) contacting the adult mammalian stem cell with a test compound; and
(c) detecting the expression of Oct-4 under conditions that normally promote differentiation and loss of Oct-4 expression in the adult mammalian stem cell,
wherein the loss of Oct-4 expression in the adult mammalian stem cell contacted with the test compound indicates that the test compound possesses anti-cancer activity.

33. The method of claim 32, further comprising detecting GJIC expression or activity in the adult mammalian stem cell, wherein the presence of GJIC expression or activity in the cells that would otherwise continue to express Oct-4 under conditions that normally promote differentiation and loss of Oct-4 expression further indicates that the test compound possesses anti-carcinogenic activity.

34. The method of claim 32, wherein the adult mammalian stem cell expressing Oct-4 is a human adult stem cell.

35. The method of claim 34, wherein the human adult stem cell is selected from the group consisting of breast stem cells, liver stem cells, pancreatic stem cells, kidney stem cells, mesenchyme stem cells and gastric stem cells.

36. A method of detecting adult mammalian stem cells comprising:
a) providing a sample comprising cells;
b) testing said cells for the presence of Oct-4 gene expression and gap junctional intercellular communication function,
wherein the presence of Oct-4 gene expression and absence of gap junctional intercellular communication function in one or more cells indicating that those cells are adult mammalian stem cells.

37. The method of claim 36, further comprising comparing the level of expression of gap junctional intercellular communication (GJIC) activity in the one or more cells in which Oct-4 gene expression is present, and in which there is an absence of gap junctional intercellular communication function, to the level of gap junctional intercellular communication (GJIC) activity in the background of other cells in the sample.

38. The method of claim 37, wherein the amount of GJIC activity is no more that two times above background.

39. The method of claim 37, wherein the amount of GJIC activity is no more than 50% of the positive control.

40. The method of claim 37, wherein the amount of GJIC activity is no more than 25% of the positive control.

41. The method of claim 37, wherein the amount of GJIC activity is no more than 10% of the positive control.

42. The method of claim 36, wherein the sample is obtained by biopsy.

43. The method of claim 36, wherein the sample comprises cells from a tissue selected from the group consisting of: breast cells, liver cells, pancreatic cells, kidney cells, mesenchyme cells and gastric cells.

44. The method of claim 36, wherein the cells in the sample of cells to be tested are lysed.

45. The method of claim 36, wherein the presence of Oct-4 gene expression is tested by a method selected from the group consisting of Western blotting, Southern blotting, Northern blotting, immunohisto-chemistry, PCR, RT-PCR, HPLC, mass spectroscopy, and enzymatic detection.

46. The method of claim 36, wherein the presence of gap junctional intercellular communication activity is tested by detecting the cell-to-cell transfer of Lucifer Yellow dye.

47. The method of claim 36, wherein the presence of Oct-4 gene expression and gap junctional intercellular communication activity are each, independently of the other, measured by a method selected from the group consisting of Western blotting, Southern blotting, Northern blotting, immunohistochemistry, PCR, RT-PCR, high pressure liquid chromatography (HPLC), mass spectroscopy, DNA chip assay and enzymatic detection assay.

48. A method of testing compounds for inhibition of cell migration comprising:
(a) providing: (i) a sample comprising cells, at least a portion of said cells characterized by the expression of Oct-4 and the absence of gap junctional intercellular communication activity; (ii) a test compound; and (iii) an assay for measuring cell migration;
(b) contacting the sample of cells with the test compound and assaying the cells for cell migration; and
(c) comparing the cell migration activity of the treated cells to the cell migration activity of an untreated control,
wherein a decrease in the cell migration activity of the treated cell s indicates that the test compound inhibits cell migration activity.

49. The method of claim 48, wherein the assaying comprises comparing the migration of the treated cells in the migration assay with one or more controls to detect inhibition of cell migration.

50. The method of claim 48, wherein the sample comprises cells characterized by an amount of GJIC activity that is no more that two times above background.

51. The method of claim 50, wherein the amount of GJIC activity is no more than 50% of the positive control.

52. The method of claim 50, wherein the amount of GJIC activity is no more than 25% of the positive control.

53. The method of claim 50, wherein the amount of GJIC activity is no more than 10% of the positive control.


SEQUENCE LISTING (8 sequences)

SEQ ID NO: 1 - DNA, 25 bp, Artificial Sequence
  Description of Artificial Sequence Synthetic primer
gacaacaatgagaaccttcaggaga

SEQ ID NO: 2 - DNA, 21 bp, Artificial Sequence
  Description of Artificial Sequence Synthetic primer
ctggcgccggttacagaacca

SEQ ID NO: 3 - DNA, 20 bp, Artificial Sequence
  Description of Artificial Sequence Synthetic primer
gttcgacagtcagccgcatc

SEQ ID NO: 4 - DNA, 21 bp, Artificial Sequence
  Description of Artificial Sequence Synthetic primer
gtgggtgtcgctgttgaagtc

SEQ ID NO: 5 - DNA, 1371 bp, Homo sapiens
ctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgaggtgcctgcccttctaggaatgggggacagggggaggggaggagctagggaaagaaaacctggagtttgtgccagggtttttggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcttgggacaca

SEQ ID NO: 6 - DNA, 3088 bp, Homo sapiens
acaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacat

SEQ ID NO: 7 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 8 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI